(19) United States
(12) Patent Application Publication
(10) Pub. No.: US 2026/0286252 A1
(43) Pub. Date: Sep. 24, 2026

(54) FLUORESCENT WHITENING AGENTS

(71) Applicant: Thomas Swan & Co. Ltd., Consett Durham (GB)

(72) Inventors: Jack Robert Ellison, Consett Durham (GB); Simon Jonathon Grant, Consett Durham (GB); Cleo Watson, Consett Durham (GB); Patrice Georges Antonin Ribiere, Consett Durham (GB); Craig Anderson, Consett Durham (GB); Rusalim-Antoniu Petris, Consett Durham (GB)

(73) Assignee: Thomas Swan & Co. Ltd., Consett Durham (GB)

(21) Appl. No.: 19/163,083

(22) PCT Filed: Mar. 7, 2024
(86) PCT No.: PCT/EP2024/056098

§ 371 Date: Sep. 8, 2025

(30) Foreign Application Priority Data
Mar. 8, 2023 (GB) 2303379.8
Mar. 8, 2023 (GB) 2303384.8
Mar. 8, 2023 (GB) 2303393.9
Jan. 26, 2024 (GB) 2401029.0

(51) Int. Cl. C11D 3/28

(52) U.S. Cl. CPC C11D 3/28

(57) ABSTRACT

The invention provides a fluorescent whitening agent or optical brightener of formula (I) or (II) having the structure: (I) (II), wherein Y is Z or X′L; wherein: A is CR¹R²(CH₂)ₙ wherein R¹ is H, CH₂OH or COZ, R² is H or CH₂OH and wherein n is 1 or 2, or wherein A is an optionally substituted aromatic or heteroaromatic ring fused with N and X wherein N and X are respectively bound to adjacent carbon atoms on the ring; X is O, S or N—H; Y is Z or X′L; the or each Z is independently OH, OM, OR³, O(CH₂)_{q}SO₃M, NH₂, NHOH, NHR⁴ or NR⁴₂, wherein M is selected from an alkali metal or an alkaline earth metal; R³ represents a linear or branched alkyl, an aryl, an alkaryl, an aralkyl, a linear or branched alkyl alcohol, a linear or branched alkyl alcohol polyol, a hydroxyalkylamine, a polyhydric alcohol, a sugar, a linear or branched alkyl ether, a polyester having from 2 to 1000 repeat units, or a polyoxyalkylene chain having from 2 to 1000 repeating units; q is 1 to 5; R⁴ is independently selected from methyl, ethyl, propyl, C₄ to C₁₂ alkyl, benzyl, 2-hydroxyethyl, 1-hydroxy-2-propanyl, 1,3-dihydroxy-2-propanyl, 2,3-dihydroxypropyl, cyanomethyl, 2-aminoethyl or dicarbamoylmethyl; alternatively, NR⁴₂ represents a heterocyclic ring, e.g. morpholine; and wherein when Y is X′L; X′ is O, S or N—H; and L represents a linker moiety connecting m repeating units of formula I or II having X′ as defined above in place of Y; R is H, CHR⁵R⁶, R⁵, R⁶, an alkylsulphonate, a polyether, CH₂COZ or CH(COZ)((CH)ₙCOZ) in which each Z may be the same or different, R⁵ and R⁶ independently represent hydrogen, alkyl (such as methyl, ethyl, propyl, isopropyl), vinyl, phenyl, hydroxymethyl, 1-hydroxyethyl, 2-hydroxyethyl, 1,2-dihydroxyethyl, aminomethyl, (dimethylamino)methyl, 5-aminopentyl, or cyano, optionally wherein R⁵ and R⁶ are the same or different, and n is 1 or 2.

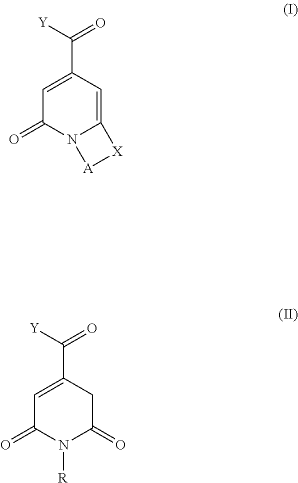

### TECHNICAL FIELD

The present invention concerns fluorescent whitening agents (FWAs), optical brighteners and certain applications.

### BACKGROUND

Industry standard FWAs in the laundry detergent industry include CAS 16090-02-1 (DAS-1), CAS 16470-24-9 (DAS-2), CAS 27344-41-8 (DSBP) and CAS 4404-43-7 (FB28). These and other conventional FWAs or optical brighteners suffer from one or more of the following disadvantages:
1. They are conventionally manufactured from fossil fuels;
2. They are non-biodegradable;
3. They are toxic in the aquatic environment.

The present invention aims to provide improved FWAs or optical brighteners in at least one of these respects.

There is prior art recognition that fluorophores may be synthesised from sustainable materials such as citric acid.

In this respect RSC Adv., 2015, 5, 34795, discloses 4-oxo-1-thia-3a-aza-6-indancarboxylic and 5-oxo-2,3-dihydro-5H-[1,3]oxazolo[3,2-a]pyridine-7-carboxylic acid via condensation of citric acid with amino acids or derivatives thereof, but with no suggestion of any potential application. The first mentioned compound is also disclosed in CN108101929 and in CN104530089 in the context of fluorescent imaging agents for medical use.

J. Mater. Chem. C, 2015, 3, 5976-5984 discloses 1,2,3,5-tetrahydro-5-oxo-imidazo[1,2-a]pyridine-7-carboxylic acid) and methyl 4-oxo-1-thia-3a-aza-6-indancarboxylate in connection with the manufacture of fluorescent carbon nanoparticles from the pyrolysis of citric acid and an amine. The first of these compounds is also disclosed for use as a pharmaceutical intermediate in CN114507230.

Methyl-4-oxo-1-thia-3a-aza-3,6-indancarboxylic acid is disclosed, also in connection with fluorescent carbon nanoparticles, in RSC Adv., 2022, 12, 19, 11640-11648 (Table 1 A-B)

CN109438479 discloses hexyl 4-oxo-1-thia-3a-aza-6-indancarboxylate and certain other indancarboxylate compounds in connection with potential application in fluorescent whitening, although the specific field of application is unspecified.

Dimeric and oligomeric indancarboxylate compounds are broadly disclosed in US2019/231909 but without any indication of application.

Fluorescent imagers such as 1-(2-Hydroxyethyl)-2,6-dioxo-1,3-dihydroisonicotinic acid and 1-oxo-1H-pyrido[2,1-b][1,3]benzothiazole-3-carboxylic acid are disclosed in WO2016/164437.

The present invention seeks to provide FWA or optical brightener with comparable functionality to the above-mentioned industry standards, but with fewer environmental drawbacks.

### SUMMARY OF THE INVENTION

According to a first aspect of the invention, there is provided a fluorescent whitening agent or optical brightener of formula (I) having the structure:

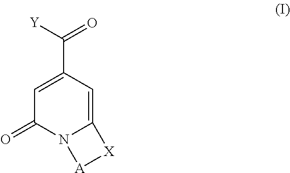

wherein:
A is CR¹R²(CH₂)ₙ wherein R¹ is H, CH₂OH or COZ, R² is H or CH₂OH and wherein n is 1 or 2, or wherein A is an optionally substituted aromatic or heteroaromatic ring fused with N and X wherein N and X are respectively bound to adjacent carbon atoms on the ring;
X is O, S or N—H;
Y is Z or X′L;
the or each Z is independently OH, OM, OR³, O(CH₂)_{q}SO₃M, NH₂, NHOH, NHR⁴ or NR⁴₂, wherein
M is selected from an alkali metal or an alkaline earth metal;
R³ represents a linear or branched alkyl, an aryl, an alkaryl, an aralkyl, a linear or branched alkyl alcohol, a linear or branched alkyl alcohol polyol, a hydroxyalkylamine, a polyhydric alcohol, a sugar, a linear or branched alkyl ether, a polyester having from 2 to 1000 repeat units, or a polyoxyalkylene chain having from 2 to 1000 repeating units;
q is 1 to 5;
the or each R⁴ is independently selected from methyl, ethyl, propyl, C₄ to C₁₂ alkyl, benzyl, 2-hydroxyethyl, 1-hydroxy-2-propanyl, 1,3-dihydroxy-2-propanyl, 2,3-dihydroxypropyl, cyanomethyl, 2-aminoethyl or dicarbamoylmethyl; alternatively, NR⁴₂ represents a heterocyclic ring, e.g. morpholine; and wherein when Y is X′L;
X′ is O, S or N—H; and
L represents a linker moiety connecting m repeating units of formula I having X′ as defined above in place of Y.

In embodiments where A is an optionally substituted heteroaromatic ring, A is preferably a pyridine ring.

In embodiments where R³ represents a linear or branched alkyl, R³ is preferably selected from methyl, ethyl, propyl, butyl, pentyl, hexyl, heptyl, octyl, or 2-ethylhexyl.

In embodiments where R³ represents an alkyl alcohol, R³ is preferably selected from methanol, ethanol, propanol, butanol, pentanol, hexanol, heptanol, octanol, dodecanol, or stearyl or oleyl alcohol.

In embodiments where R³ represent an aryl, R³ is preferably phenyl, and in embodiments where R³ represents alkaryl, R³ is preferably selected from benzyl, or ethyl phenyl.

In embodiments where R³ represents a linear or branched alkyl alcohol, R³ is preferably selected from 4-hydroxybutyl.

In embodiments where R³ represents a linear or branched alcohol polyol, R³ is preferably a diol or triol. In most preferable instances, R³ may be selected from 2,3-dihydroxypropyl, or 2-hydroxy-1-(hydroxymethyl)ethyl.

In embodiments where R³ represents a hydroxyalkylamine, R³ is preferably selected from triethanolamine, N-methyldiethanolamine or triisopropanolamine.

In embodiments where R³ represents a polyhydric alcohol, R³ is preferably glycerol.

In embodiments where R³ represents a sugar, R³ is preferably selected from dextrose, fructose, galactose, glucose, lactose, maltose or sucrose.

In embodiments where R³ represents a linear or branched alkyl ether, R² is preferably selected from 2-(2-hydroxyethoxy)ethyl, 2-(2-hydroxy-1-methylethoxy)-1-methylethyl, 2-[2-(2-hydroxy-1-methylethoxy)-1-methylethoxy]-1-methylethyl, 3-(3-hydroxypropoxy)propyl, 3-[3-(3-hydroxypropoxy)propoxy]propyl, 4-(4-hydroxybutoxy)butyl, 4-[4-(4-hydroxybutoxy)butoxy]butyl, 2-(2-methoxyethoxy)ethyl, or 2-[2-(2-methoxyethoxy) ethoxy]ethyl.

In embodiments where R³ represents a polyester or a polyoxyalkylene having from 2 to 1000 repeat units, it may be a homopolymer or a copolymer.

Linker L may be derived from an alcohol, in which case X′ is the oxygen of the precursor alcohol; or may be derived from an amine, in which case X′ is the nitrogen of the precursor amine.

In dimeric or oligomeric compounds according to the invention, m is 2 or more, preferably 3 to 20, most preferably 3.

Linker L may be constituted by any of ethylene, propylene or C₄ to C₁₂ alkylene, a polyester chain having from 0 to 1000 repeating units being a homopolymer or copolymer, or a polyoxyalkylene chain having from 0 to about 1000 repeating units being a homopolymer or copolymer, triethanolamine, glycerol or a sugar.

According to a second aspect of the invention, there is provided a fluorescent whitening agent or optical brightener of formula (II) having the structure:

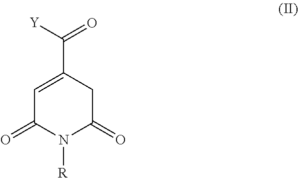

wherein Y is Z or X′L;
   Z is OH, OM, OR³, O(CH₂)_{q}SO₃M, NH₂, NHOH, NHR⁴, or NR⁴₂,
   M represents an alkali metal or an alkaline earth metal;
   R³ represents a linear or branched alkyl, an aryl, an alkaryl, an aralkyl, a linear or branched alkyl alcohol, a linear or branched alcohol polyol, a hydroxyalkylamine, a polyhydric alcohol, a sugar, a linear or branched alkyl ether, a polyester having from 2 to 1000 repeat units, or a polyoxyalkylene chain having from 2 to 1000 repeating units;
   q is 1 to 5;
   R⁴ is independently selected from methyl, ethyl, propyl, C₄ to C₁₂ alkyl, benzyl, 2-hydroxyethyl, 1-hydroxy-2-propanyl, 1,3-dihydroxy-2-propanyl, 2,3-dihydroxypropyl, cyanomethyl, 2-aminoethyl or dicarbamoylmethyl; alternatively, NR⁴₂ represents a heterocyclic ring, e.g. morpholine; and
R is H, CHR⁵R⁶, R⁵, R⁶, an alkylsulphonate, a polyether, CH₂COZ or CH(COZ)((CH)ₙCOZ) in which each Z may be the same or different,
   R⁵ and R⁶ independently represent hydrogen, alkyl (such as methyl, ethyl, propyl, isopropyl), vinyl, phenyl, hydroxymethyl, 1-hydroxyethyl, 2-hydroxyethyl, 1,2-dihydroxyethyl, aminomethyl, (dimethylamino)methyl, 5-aminopentyl, or cyano, optionally wherein R⁴ and R⁵ are the same or different, and
   n is 1 or 2;
wherein when Y is X′L;
   X′ is O, S or N—H; and
   L represents a linker moiety connecting m repeating units of a variant of formula II in which X′ as defined above is present in place of Y.

In embodiments where R³ represents a linear or branched alkyl, R³ is preferably selected from methyl, ethyl, propyl, butyl, pentyl, hexyl, heptyl, octyl, or 2-ethylhexyl.

In embodiments where R³ represents an alkyl alcohol, R³ is preferably selected from methanol, ethanol, propanol, butanol, pentanol, hexanol, heptanol, octanol, dodecanol, or stearyl or oleyl alcohol.

In embodiments where R³ represent an aryl, R³ is preferably phenyl, and in embodiments where R³ represents an alkaryl, R³ is preferably selected from benzyl, or ethyl phenyl.

In embodiments where R³ represents a linear or branched alkyl alcohol, R³ is preferably selected from 4-hydroxybutyl.

In embodiments where R³ represents a linear or branched alcohol polyol, R³ is preferably a diol or triol. In most preferable instances, R³ may be selected from 2,3-dihydroxypropyl, or 2-hydroxy-1-(hydroxymethyl)ethyl.

In embodiments where R³ represents a hydroxyalkylamine, R³ is preferably selected from triethanolamine, N-methyldiethanolamine or triisopropanolamine.

In embodiments where R³ represents a polyhydric alcohol, R³ is preferably glycerol.

In embodiments where R³ represents a sugar, R³ is preferably selected from dextrose, fructose, galactose, glucose, lactose, maltose or sucrose.

In embodiments where R³ represents a linear or branched alkyl ether, R³ is preferably selected from 2-(2-hydroxyethoxy)ethyl, 2-(2-hydroxy-1-methylethoxy)-1-methylethyl, 2-[2-(2-hydroxy-1-methylethoxy)-1-methylethoxy]-1-methylethyl, 3-(3-hydroxypropoxy)propyl, 3-[3-(3-hydroxypropoxy)propoxy]propyl, 4-(4-hydroxybutoxy)butyl, 4-[4-(4-hydroxybutoxy)butoxy]butyl, 2-(2-methoxyethoxy)ethyl, or 2-[2-(2-methoxyethoxy) ethoxy]ethyl.

In embodiments where R³ represents a polyester or a polyoxyalkylene chain having from 2 to 1000 repeat units, it may be a homopolymer or a copolymer.

Linker L may be derived from an alcohol, in which case X′ is the oxygen of the precursor alcohol; or may be derived from an amine, in which case X′ is the nitrogen of the precursor amine.

In dimeric or oligomeric compounds according to the invention, m is 2 or more, preferably 3 to 20, most preferably 3.

Linker L may be constituted by any of ethylene, propylene or C₄ to C₁₂ alkylene, a polyester chain having from 0 to 1000 repeating units being a homopolymer or copolymer, or a polyoxyalkylene chain having from 0 to about 1000 repeating units being a homopolymer or copolymer, triethanolamine, glycerol or a sugar.

The FWAs and optical brighteners of the invention are preferably formulated for use as fluorescent whitening agents, or optical brighteners, as the case may be. Typically, such formulation involves the provision of the active ingredient (FWA or optical brightener) in conjunction with at least one ancillary compound typically present in fluorescent whitening or optical brightening formulations. Such ancillary compounds may be selected from, for example, one or more of detergent(s), bleach(es), carrier compound(s), stabilizer(s) and/or dispersant(s).

The invention also provides the use of the aforementioned compounds as fluorescent whitening agents or optical brighteners.

The invention also provides a fluorescent whitening or optical brightening formulation comprising the aforesaid fluorescent whitening agent or optical brightener and at least one detergent, bleach, carrier compound, stabilizer and/or dispersant.

Also provided in accordance with the invention is a method for providing fluorescent whitening or optical brightening to a substrate comprising contacting the substrate with one or more of the aforementioned compounds under conditions effective to allow chemical and/or physical bonding of the one or more compounds onto or into the substrate.

### DETAILED DESCRIPTION

Current industry standard fluorescent whitening agents or optical brighteners, including DAS-1, DAS-2, DSPB and FB28, all absorb ultraviolet light in the region 340-370 nm and emit visible blue light in the region 420-470 nm. To achieve this effect, elongated conjugated molecular systems are required to provide a suitable number of π-electrons in the conjugated aromatic system, to enable a suitable energy transition that achieves emission at 420-470 nm upon de-excitation. As demonstrated by the chemical structures of the current industry standards, this typically requires elongated aromatic systems based predominantly on carbon and hydrogen, which have high molecular weights.

Pyridone and citrazinic acid derivatives are known to achieve similar π-electron counts and also emit visible blue light in the 420-470 nm range. These derivatives, which have substantially lower molecular weights, achieve this through the incorporation of heteroatoms, such as nitrogen and oxygen.

The excitation and de-excitation properties of the fluorescent whitening agents or optical brighteners of the invention may be mechanistically understood with reference to prior art studies SC Adv., 2015, 5, 34795 attributes the fluorescence in the 420-470 nm range of similar compounds to π*-π transitions in the same carbon-oxygen double bond, as is found in scaffolds based on pyridone and citrazinic acid. The carboxyl groups, particularly the carboxylic acid groups, present in the structures of formula (I) and (II) of the present invention are believed to contribute to the required number of π-electrons associated with fluorescence in the desired range.

As a result, the fluorescent whitening agents or optical brighteners of the invention are advantageous over current industry standards as they provide comparable fluorescence using a smaller compound, with a lower molecular weight. Additionally, further functionalisation of the structures of formulae (I) and (II) to modulate application performance may be readily achieved, with little effect on the fluorescent behaviour of the fluorescent whitening agent or optical brightener.

There are several further factors governing the efficacy of FWA or optical brightener according to the invention.

The FWA or optical brightener according to the invention typically absorb ultraviolet light in the region 340-370 nm and emit visible light in the region 420-470 nm. The quantum yield of these compounds in this respect is preferably at least about 30%, more preferably at least about 40%, still more preferably at least about 50%, most preferably at least about 60%.

The FWAs or optical brighteners of the present invention exhibit much higher relative quantum yields than current industry standards. For example, only DSPB gives a comparable value of greater than 70%. The FWAs or optical brighteners described in the present invention are advantageous over current standards, as a similar fluorescence performance can be achieved with a much smaller molecular weight, which in turn can lead to a lower loading of the FWA or optical brightener in the final application. Lower loadings can mitigate any potential adverse effects associated with the compound.

The FWAs or optical brighteners of the invention typically exhibit good substantivity onto cellulosic fabrics, retaining at least about 30% loading after a typical wash cycle.

FWAs or optical brighteners in accordance with the invention can be sustainably produced from readily available starting materials, using environmentally sustainable reagents. Therefore, also provided in accordance with the invention is a fluorescent whitening agent or optical brightener derived from the condensation of a di- or polycarboxylic acid and an amine—in particular, between a di- or polycarboxylic acid and an amino acid or amino acid derivative—in particular between citric acid and an amino acid or amino acid derivative.

Current industry standard FWAs and optical brighteners, including DAS-1, DAS-2, DSPB and FB28, have large conjugated aromatic systems of high molecular mass, which have limited capability to be oxidised or hydrolysed, making them less biodegradable. As a result, current industry standard FWAs are a raising environmental concern as they are not only detected as pollutants in urban wastewaters (Marine Pollution Bulletin, 2022, 178, 113559) but also in indoor environments (Environ. Sci. Technol., 2022, 56, 10131-10140). Recent studies have identified that FWAs are a source of pollution in urban wastewater, which negatively impacts aquatic ecosystems and water resources. The Marine Pollution Bulletin, 2022, 178, 113559 identifies FWAs as a pollutant of emerging concern, as they are present in wastewater in concentrations comparable to the top range of more well-studied wastewater pollutants, such as pharmaceuticals (approx. 10-1000 ng/L). Data from Sweden showed that DAS-2 was always detected in sample effluents in high median concentrations of approximately 1500 ng/L.

In comparison, the FWAs and optical brighteners in accordance with the invention have smaller fluorescent cores and are thus more biodegradable as they are more prone to biological attacks, whilst still providing enough stability to oxidation and hydrolysis to be of use in the intended applications.

The molecular weight of the FWAs and optical brighteners of the present invention is preferably below about 1000, below about 750, below about 500, below about 400, below about 300, or below about 200.

Estimation of biodegradation based on chemical structures is complex due to variations in the physicochemical properties of organic compounds. Predictive methods for estimating biodegradation previously relied on metabolic pathways and on microbial diversity. More recently, predictive methods use group contribution, QSAR, or machine learning methods.

As detailed in Ecotoxicol. Environ. Saf. 1989, 18, 252-267, from a survey conducted with 22 biodegradation experts, a hierarchy was constructed to represent the approximate order in which various groups were viewed as contributing to aerobic biodegradability: ester=amide=anhydride>hydroxyl>carboxylic acid=epoxide=site of unsaturation>benzene ring=methyl=methylene group. The general inference was that compounds already partially oxidised were generally considered to be more prone to biological attack than those which were not partially oxidised, all other things being equal, and that hydrolysable chemicals are considered to be more easily degraded still. The time taken for ultimate degradation was generally deduced from consideration of molecular weight, branching, halogenation, functional groups, solubility and other factors.

Advantageously, the FWAs and optical brighteners in accordance with the invention are biodegradable. Preferably they contain at least one functional group which is not oxidised or which is only partially oxidised, and thereby is oxidisable or further oxidisable. Preferably they contain at least one hydrolysable group.

Therefore, also provided in accordance with the invention is an FWA or optical brightener comprising at least one labile group. Inherently biodegradable fluorescent whitening agents may be defined as >20% but <60% biodegradability in water as measured by standard OECD 301A-F testing. A readily biodegradable fluorescent whitening agent or optical brightener may be characterised by the ability of the material to biodegrade quickly and completely in water (either ≥70% dissolved organic carbon removal, ≥60% theoretical carbon dioxide or ≥60% theoretical oxygen demand, depending on standard OECD 301A-F test methods) in a 10-day window within 28 days.

FWAs and optical brighteners in accordance with the invention are preferably stable at relatively high pH, for example from about pH 7 to about pH 10, pH 7.5 to about 9.5, typical conditions in a laundry wash.

FWAs and optical brighteners in accordance with the invention are preferably UV stable.

FWAs and optical brighteners in accordance with the invention are preferably oxidatively stable in a conventional wash cycle.

In accordance with the invention, preferred examples of the fluorescent whitening agents and optical brighteners are:

| Codes | Structure and Description |
|---|---|
| 1a | 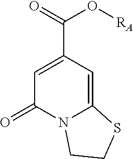 |
| | In embodiments of |
| | general formula 1a, |
| | R_{A} may be an alkyl |
| | group, such as for |
| | example methyl, ethyl, |
| | propyl, butyl, pentyl, |
| | hexyl, heptyl, octyl, a |
| | branched alkyl group, |
| | such as for example |
| | isopropyl, 2-ethylhex- |
| | yl,or a benzyl group. |
| 1b | 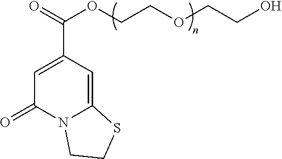 |
| | In embodiments of general formula 1b, the |
| | ester may be the ester of an alkyl alcohol, |
| | comprising a polyoxyalkylene chain having |
| | repeating units, such as for example a poly- |
| | oxyethylene chain where n is from 2-20. |
| 1c | 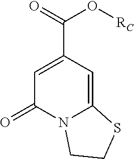 |
| | In embodiments of |
| | general formula 1c, R_{C} |
| | may be an alkyl |
| | sulphonate or sulphonic |
| | acid of general formula |
| | (CH₂)_{q}SO₃M, wherein |
| | q is 1-5 and M is H or |
| | a metal, such as for |
| | example methane |
| | sulphonic acid or |
| | ethane sulphonic acid. |
| 1d | 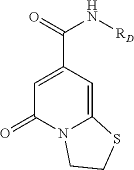 |
| | In embodiments of |
| | general formula 1d, R_{D} |
| | may be an alkyl group, |
| | such as for example |
| | butyl, pentyl, hexyl, |
| | heptyl, or octyl, or |
| | R_{D} may be an alcohol, |
| | such as for example 2- |
| | hydroxyethyl, 2- |
| | hydroxy-1-(hydroxy- |
| | methyl)ethyl, or 2,3- |
| | dihydroxypropyl. |
| 2a | 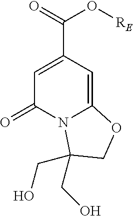 |
| | In embodiments of |
| | general formula 2a, R_{E} |
| | may be an alkyl group, |
| | such as for example |
| | methyl, ethyl, propyl, |
| | butyl, pentyl, hexyl, |
| | heptyl, octyl, a |
| | branched alkyl group, |
| | such as for example |
| | isopropyl, 2-ethylhex- |
| | yl, or a benzyl group. |
| 2b | 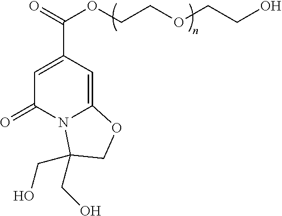 |
| | In embodiments of general formula 2b, the ester |
| | may be the ester of an alkyl alcohol, comprising |
| | a polyoxyalkylene chain having repeating |
| | units, such as for example a polyoxyethylene |
| | chain where n is from 2-20. |
| 2c | 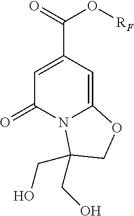 |
| | In embodiments of |
| | general formula 2c, R_{F} |
| | may be an alkyl sul- |
| | phonate or sulphonic |
| | acid of general formula |
| | (CH₂)_{q}SO₃M, wherein |
| | q is 1-5 and M is H or |
| | a metal, such as for |
| | example methane sul- |
| | phonic acid or ethane |
| | sulphonic acid. |
| 2d | 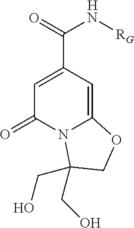 |
| | In embodiments of |
| | general formula 2d, R_{G} |
| | may be an alkyl group, |
| | such as for example |
| | butyl, pentyl, hexyl, |
| | heptyl, or octyl, or R_{G} |
| | may be an alcohol, |
| | such as for example |
| | 2-hydroxyethyl, 2- |
| | hydroxy-1-(hydroxy |
| | methyl)ethyl, or 2,3- |
| | dihydroxypropyl. |
| 3a | 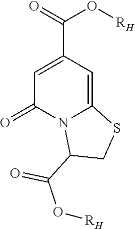 |
| | In embodiments of |
| | general formula 3a, |
| | the group in each |
| | position R_{H} may be |
| | independently hydro- |
| | gen or an alkyl, such |
| | as for example meth- |
| | yl, ethyl, propyl, butyl, |
| | pentyl, hexyl, heptyl, |
| | octyl, a branched alk- |
| | yl group, such as for |
| | example isopropyl, |
| | 2-ethylhexyl, or a |
| | benzyl group. |
| 3b | 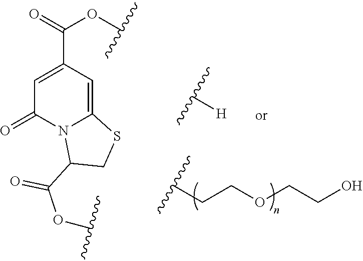 |
| | In embodiments of general formula 3b, the pendant group |
| | may be independently hydrogen or the ester of an alkyl |
| | alcohol, comprising a polyoxyalkylene chain having |
| | repeating units, such as for example a polyoxyethylene |
| | chain where n is from 2-20. |
| 3c | 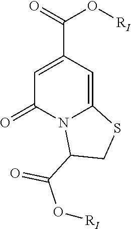 |
| | In embodiments of |
| | general formula 3c, |
| | the group in each |
| | position R_{I} may be |
| | independently |
| | hydrogen or an |
| | alkyl sulphonate |
| | or sulphonic acid |
| | of general formula |
| | —(CH₂)_{q}SO₃M, |
| | wherein q is 1-5 |
| | and M is H or a |
| | metal, such as for |
| | example methane |
| | sulphonic acid or |
| | ethane sulphonic |
| | acid. |
| 3d | 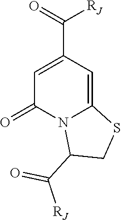 |
| | In embodiments of |
| | general formula 3d, |
| | the group in each |
| | position R_{J} may be |
| | independently a |
| | hydroxyl group or |
| | an alkyl amine group |
| | (—NH—R), such as |
| | for example butyl- |
| | amine, pentylamine, |
| | hexylamine, heptyl- |
| | amine, or octylamine, |
| | or each R_{J} may be |
| | independently a |
| | hydroxyl group or |
| | an amino alcohol |
| | (—NH—R—OH), |
| | such as for example |
| | 2-hydroxyethyl- |
| | amine, 2-hydroxy- |
| | 1-(hydroxymethyl)- |
| | ethylamine, or |
| | 2,3-dihydroxyprop- |
| | yl amine. |
| 4a | 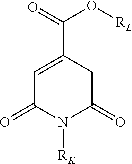 |
| | In embodiments of |
| | general formula 4a, R_{K} |
| | may be ethylalcohol |
| | (—CH₂CH₂OH), |
| | isopropylalcohol |
| | (—CH(CH₂OH)₂), |
| | 2-hydroxypropyl |
| | (—CH₂CH(OH)CH₃), |
| | benzyl (—CH₂C₆H₅), |
| | N-N-dimethylethyl |
| | (—CH₂CH₂N(CH₃)₂), |
| | propenyl |
| | (—CH₂CHCH₂), propyl |
| | (—CH₂CH₂CH₃) or |
| | ethylsulphonate |
| | (—CH₂CH₂SO₃Na); |
| | and R_{L} may be |
| | hydrogen, an alkyl |
| | group, such as for |
| | example methyl, |
| | ethyl, propyl, butyl, |
| | pentyl, hexyl, heptyl, |
| | octyl, a branched |
| | alkyl group, such as |
| | for example isoprop- |
| | yl, 2-ethylhexyl, or |
| | a benzyl group, or R_{L} |
| | may be an alcohol or |
| | branched alcohol, such |
| | as for example 2- |
| | hydroxyethyl, 2- |
| | hydroxy-1-(hydroxy- |
| | methyl)ethyl, or 2,3- |
| | dihydroxypropyl, or |
| | R_{L} may be 2-(2- |
| | hydroxy-1-methyl- |
| | ethoxy)-1-methyl- |
| | ethyl, 2-[2-(2- |
| | hydroxy-1-methyl- |
| | ethoxy)-1-methyl- |
| | ethoxy]-1-methyl |
| | ethyl, 3-(3-hydroxy- |
| | propoxy)propyl, 3- |
| | [3-(3-hydroxy- |
| | propoxy)propoxy] |
| | propyl, 4-(4-hydroxy- |
| | butoxy)butyl, 4-[4- |
| | (4-hydroxybutoxy)- |
| | butoxy]butyl, 2-(2- |
| | methoxyethoxy)ethyl, |
| | or 2-[2-(2-methoxy |
| | ethoxy)ethoxy]ethyl. |
| 4b | 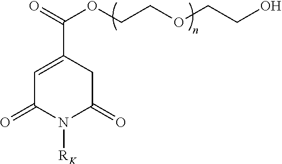 |
| | In embodiments of general formula 4b, R_{K} may |
| | be ethylalcohol (—CH₂CH₂OH), isopropyl- |
| | alcohol (—CH(CH₂OH)₂), 2-hydroxypropyl |
| | (—CH₂CH(OH)CH₃), benzyl (—CH₂C₆H₅), |
| | N-N-dimethylethyl (—CH₂CH₂N(CH₃)₂), |
| | propenyl (—CH₂CHCH₂), propyl |
| | (—CH₂CH₂CH₃) or ethylsulphonate |
| | (—CH₂CH₂SO₃Na); and the ester may be |
| | the ester of an alkyl alcohol, comprising a |
| | polyoxyalkylene chain having repeating |
| | units, such as for example a polyoxyeth- |
| | ylene chain where n is from 2-20. |
| 4c | 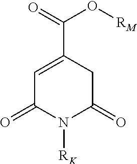 |
| | In embodiments of |
| | general formula 4c, R_{K} |
| | may be ethylalcohol |
| | (—CH₂CH₂OH), |
| | isopropylalcohol |
| | (—CH(CH₂OH)₂), |
| | 2-hydroxypropyl |
| | (—CH₂CH(OH)CH₃), |
| | benzyl (—CH₂C₆H₅), |
| | N-N-dimethylethyl |
| | (—CH₂CH₂N(CH₃)₂), |
| | propenyl |
| | (—CH₂CHCH₂), |
| | propyl (—CH₂CH₂CH₃) |
| | or ethylsulphonate |
| | (—CH₂CH₂SO₃Na); |
| | and R_{M} may be an alkyl |
| | sulphonate or sulphonic |
| | acid of general formula |
| | (CH₂),SO₃M, wherein |
| | q is 1-5 and M is H or |
| | a metal, such as for |
| | example methane |
| | sulphonic acid or |
| | ethane sulphonic acid. |
| 4d | 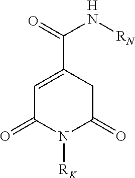 |
| | In embodiments of |
| | general formula 4d, R_{K} |
| | may be ethylalcohol |
| | (—CH₂CH₂OH), |
| | isopropylalcohol |
| | (—CH(CH₂OH)₂), |
| | 2-hydroxypropyl |
| | (—CH₂CH(OH)CH₃), |
| | benzyl (—CH₂C₆H₅), |
| | N-N-dimethylethyl |
| | (—CH₂CH₂N(CH₃)₂), |
| | propenyl |
| | (—CH₂CHCH₂), |
| | propyl (—CH₂CH₂CH₃) |
| | or ethylsulphonate |
| | (—CH₂CH₂SO₃Na); |
| | and R_{N} may be an |
| | alkyl group, such as for |
| | example butyl, pentyl, |
| | hexyl, heptyl, or octyl, |
| | or R_{N} may be an |
| | alcohol, such as for |
| | example 2-hydroxy- |
| | ethyl, 2-hydroxy-1- |
| | (hydroxymethyl)- |
| | ethyl, or 2,3- |
| | dihydroxypropyl. |
| 4e | 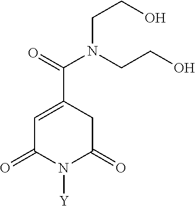 |
| | In a particular embodiment of |
| | general formula 4e, Y may be |
| | ethylalcohol (—CH₂CH₂OH), |
| | isopropylalcholol |
| | (—CH(CH₂OH)₂), 2-hydroxy- |
| | propyl (—CH₂CH(OH)CH₃), |
| | benzyl (—CH₂C₆H₅), N-N- |
| | dimethylethyl |
| | (—CH₂CH₂N(CH₃)₂), propenyl |
| | (—CH₂CHCH₂), propyl |
| | (—CH₂CH₂CH₃) or ethyl- |
| | sulphonate (—CH₂CH₂SO₃Na); |
| | and the NH group is further |
| | substituted. |
| 5a | 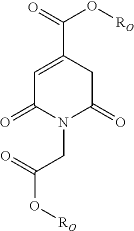 |
| | In embodiments of |
| | general formula 5a, |
| | the group in each |
| | position R_{O} may be |
| | independently |
| | hydrogen, an alkyl |
| | group, such as for |
| | example methyl, ethyl, |
| | propyl, butyl, pentyl, |
| | hexyl, heptyl, octyl, |
| | a branched alkyl |
| | group, such as for |
| | example isopropyl, |
| | 2-ethylhexyl, or a |
| | benzyl group, or the |
| | group in each position |
| | R_{O} may be an alcohol |
| | or branched alcohol, |
| | such as for example |
| | 2-hydroxyethyl, 2- |
| | hydroxy-1-(hydroxy |
| | methyl)ethyl, or 2,3- |
| | dihydroxypropyl. |
| 5b | 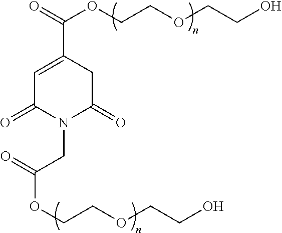 |
| | In embodiments of general formula 5b, each |
| | ester may be independently the ester of an |
| | alkyl alcohol, comprising a polyoxyalkylene |
| | chain having repeating units, such as for |
| | example a polyoxyethylene chain where n |
| | is from 2-20. |
| 5c | 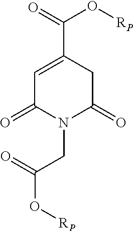 |
| | In embodiments of |
| | general formula 5c, |
| | the group in each |
| | position R_{P} may be |
| | independently an |
| | alkyl sulphonate or |
| | sulphonic acid of |
| | general formula |
| | (CH₂)_{q}SO₃M, wherein |
| | q is 1-5 and M is H |
| | or a metal, such as |
| | for example methane |
| | sulphonic acid or |
| | ethane sulphonic acid. |
| 5d | 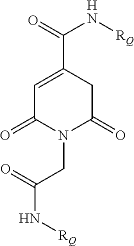 |
| | In embodiments of |
| | general formula 5d, |
| | the group in each |
| | position R_{O} may be |
| | independently an |
| | alkyl group, such as |
| | for example butyl, |
| | pentyl, hexyl, heptyl, |
| | or octyl, or the group |
| | in each position R_{O} |
| | may be an alcohol, |
| | such as for example |
| | 2-hydroxyethyl, 2- |
| | hydroxy-1-(hydroxy- |
| | methyl)ethyl, or 2,3- |
| | dihydroxypropyl. |
| 6a | 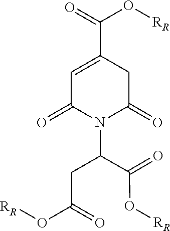 |
| | In embodiments of general |
| | formula 6a, the group in |
| | each position R_{R} may be |
| | independently hydrogen, |
| | an alkyl group, such as for |
| | example methyl, ethyl, |
| | propyl, butyl, pentyl, |
| | hexyl, heptyl, octyl, a |
| | branched alkyl group, |
| | such as for example |
| | isopropyl, 2-ethylhexyl, |
| | or a benzyl group, or the |
| | group in each position |
| | R_{R} may be an alcohol or |
| | branched alcohol, such as |
| | for example 2-hydroxy- |
| | ethyl, 2-hydroxy-1- |
| | (hydroxymethyl)ethyl, or |
| | 2,3-dihydroxypropyl. |
| 6b | 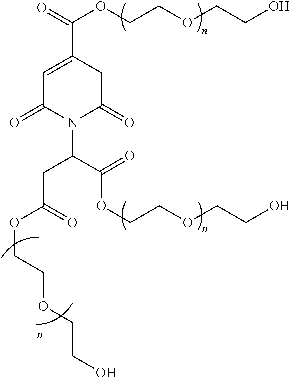 |
| | In embodiments of general formula 6b, each |
| | ester may be independently the ester of an alkyl |
| | alcohol, comprising a polyoxyalkylene chain |
| | having repeating units, such as for example a |
| | polyoxyethylene chain where n is from 2-20. |
| 6c | 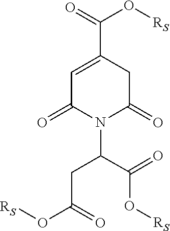 |
| | In embodiments of general |
| | formula 6c, the group in |
| | each position R_{S} may be |
| | independently an alkyl |
| | sulphonate or sulphonic |
| | acid of general formula |
| | (CH₂)_{q}SO₃M, wherein q |
| | is 1-5 and M is H or a |
| | metal, such as for example |
| | methane sulphonic acid |
| | or ethane sulphonic acid. |
| 6d | 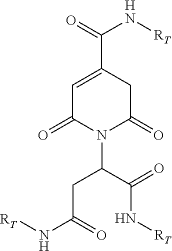 |
| | In embodiments of general |
| | formula 6d, the group in each |
| | position R_{T} may be |
| | independently an alkyl |
| | group, such as for example |
| | butyl, pentyl, hexyl, heptyl, |
| | or octyl, or the group in |
| | each position RT may be an |
| | alcohol, such as for example |
| | 2-hydroxyethyl, 2-hydroxy- |
| | 1-(hydroxymethyl)ethyl, or |
| | 2,3-dihydroxypropyl. |
| 7a | 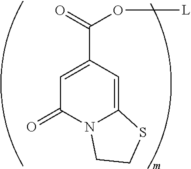 |
| | In embodiments of general |
| | formula 7a, the linker group L |
| | may be selected from any of |
| | the following alkyl, such as for |
| | example propyl, butyl, pentyl, |
| | hexyl, heptyl, octyl, or branched |
| | linkers such as, 2-hydroxyethyl |
| | amino-diethane, 3-hydroxy- |
| | propyl, or 2-hydroxypropyl. |
| 7b | 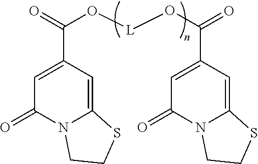 |
| | In embodiments of general formula 7b, the |
| | linker group L may be selected from straight |
| | chain or branched alkyl or alcohol groups, |
| | such as for example ethyl, 1-methylethyl, |
| | propyl, 2-methylpropyl, 2-hydroxypropyl, |
| | butyl, and the number of repeat units, n, |
| | may be selected from 2-9. |
| 7c | 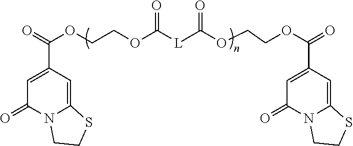 |
| | In embodiments of general formula 7c, the linker group L |
| | may be selected from,ethyl, propyl, benzyl, furanyl; and |
| | the number of repeat units, n, may be selected from |
| | 2-10. |
| 7d | 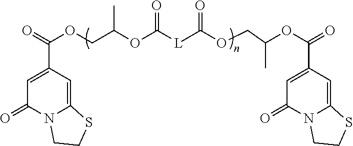 |
| | In embodiments of general formula 7d, the linker group |
| | L may be selected from, ethyl, propyl, benzyl, furanyl; |
| | and the number of repeat units, n, may be selected from |
| | 2-10. |
| 7e | 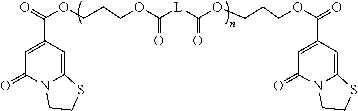 |
| | In embodiments of general formula 7e, the linker group |
| | L may be selected from, ethyl, propyl, benzyl, furanyl; |
| | and the number of repeat units, n, may be selected from |
| | 2-10. |
| 7f | 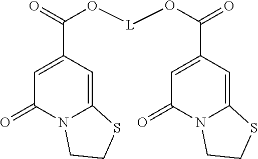 |
| | In specific embodiments of general |
| | formula 7f, the linker group L may be |
| | selected from straight chain, branched |
| | alkyl or alcohol chains, or hetero |
| | cyclic rings, or a combination thereof. |
| 8a | 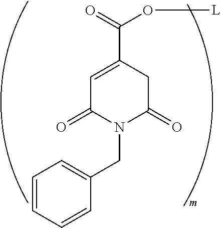 |
| | In embodiments of general formula |
| | 8a, the linker group L may be |
| | selected from any of the following |
| | alkyl, such as for example propyl, |
| | butyl, pentyl, hexyl, heptyl, octyl, |
| | or branched linkers such as, 2- |
| | hydroxyethylamino-diethane, 3- |
| | hydroxypropyl, or 2-hydroxypropyl. |
| 8b | 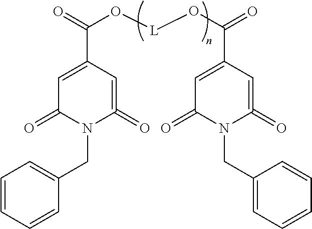 |
| | In embodiments of general formula 8b, the linker |
| | group L may be selected from straight chain or |
| | branched alkyl or alcohol groups, such as for |
| | example ethyl, 1-methylethyl, propyl, 2-methyl- |
| | propyl, 2-hydroxypropyl, butyl, and the number |
| | of repeat units, n, may be selected from 2-9. |
| 8c | 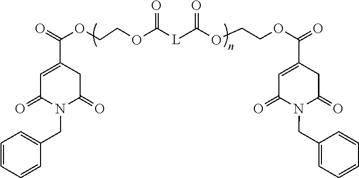 |
| | In embodiments of general formula 8c, the linker group |
| | L may be selected from, ethyl, propyl, benzyl, furanyl; |
| | and the number of repeat units, n, may be selected from |
| | 2-10. |
| 8d | 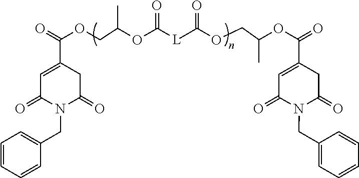 |
| | In embodiments of general formula 8d, the linker group L |
| | may be selected from, ethyl,propyl, benzyl, furanyl; and |
| | the number of repeat units, n, may be selected from 2-10. |
| 8e | 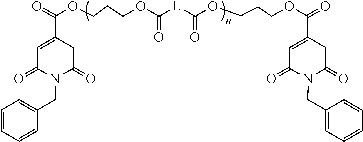 |
| | In embodiments of general formula 8e, the linker group L |
| | may be selected from, ethyl, propyl, benzyl, furanyl; and |
| | the number of repeat units, n, may be selected from 2-10. |
| 8f | 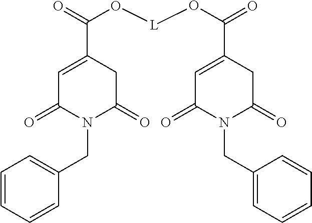 |
| | In specific embodiments of general formula 8f, the |
| | linker group L may be selected from straight chain, |
| | branched alkyl or alcohol chains, or heterocyclic |
| | rings, or a combination thereof. |
| 9a | 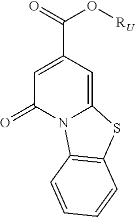 |
| | In embodiments of |
| | general formula 9a, R_{U} |
| | may be hydrogen or an |
| | alkyl group, such as for |
| | example methyl, ethyl, |
| | propyl, butyl, pentyl, |
| | hexyl, heptyl, octyl, a |
| | branched alkyl group, |
| | such as for example |
| | isopropyl, 2-ethylhexyl, |
| | or a benzyl group. |
| 9b | 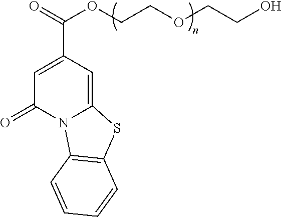 |
| | In embodiments of general formula 9b, the ester |
| | may be the ester of an alkyl alcohol, comprising |
| | a polyoxyalkylene chain having repeating units, |
| | such as for example a polyoxyethylene chain |
| | where n is from 2-20. |
| 9c | 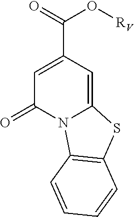 |
| | In embodiments of |
| | general formula 9c, |
| | R_{Y} may be an alkyl |
| | sulphonate or sulphonic |
| | acid of general formula |
| | (CH₂)_{q}SO₃M, wherein |
| | q is 1-5 and M is H or |
| | a metal, such as for |
| | example methane |
| | sulphonic acid or |
| | ethane sulphonic acid. |
| 9d | 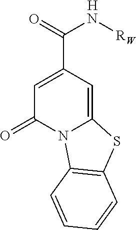 |
| | In embodiments of |
| | general formula 9d, |
| | R_{W} may be an alkyl |
| | group, such as for |
| | example butyl, pentyl, |
| | hexyl, heptyl, or octyl, |
| | or R_{W} may be an |
| | alcohol, such as for |
| | example 2-hydroxy- |
| | ethyl, 2-hydroxy-1- |
| | (hydroxymethyl)ethyl, |
| | or 2,3-dihydroxypropyl. |
| 10a | 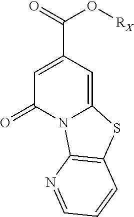 |
| | In embodiments of |
| | general formula 10a, |
| | R_{X} may be hydrogen |
| | or an alkyl group, such |
| | as for example methyl, |
| | ethyl, propyl, butyl, |
| | pentyl, hexyl, heptyl, |
| | octyl, a branched alkyl |
| | group, such as for |
| | example isopropyl, |
| | 2-ethylhexyl, or a |
| | benzyl group; and X |
| | may be selected from |
| | is O, S or NH. The |
| | embodiment shown |
| | shows N in position |
| | 3. However, in other |
| | embodiments N may |
| | be in position 4, 5 or |
| | 6 ofthe aromatic ring. |
| 10b | 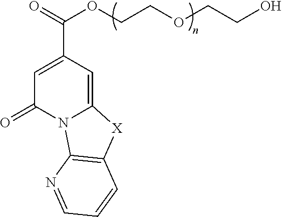 |
| | In embodiments of general formula 10b, the |
| | ester may be the ester of an alkyl alcohol, |
| | comprising a polyoxyalkylene chain having |
| | repeating units, such as for example a poly- |
| | oxyethylene chain where n is from 2-20; and |
| | X may be selected from is O, S or NH. The |
| | embodiment shown shows N in position 3. |
| | However, in other embodiments N may be |
| | in position 4, 5 or 6 of the aromatic ring. |
| 10c | 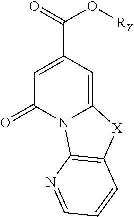 |
| | In embodiments of |
| | general formula 10c, |
| | R_{Y} may be an alkyl |
| | sulphonate or sulphonic |
| | acid of general formula |
| | (CH₂)_{q}SO₃M, wherein |
| | q is 1-5 and M is H or |
| | a metal, such as for |
| | example methane |
| | sulphonic acid or |
| | ethane sulphonic acid; |
| | and X may be selected |
| | from is O, S or NH. |
| | The embodiment shown |
| | shows N in position 3. |
| | However, in other |
| | embodiments N may |
| | be in position 4, 5 or |
| | 6 of the aromatic ring. |
| 10d | 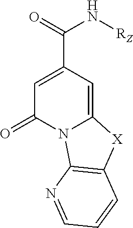 |
| | In embodiments of |
| | general formula 10d, |
| | R_{Z} may be an alkyl |
| | group, such as for |
| | example butyl, pentyl, |
| | hexyl, heptyl, or octyl, |
| | or RZ may be an |
| | alcohol, such as for |
| | example 2-hydroxy- |
| | ethyl, 2-hydroxy-1- |
| | (hydroxymethyl)ethyl, |
| | or 2,3-dihydroxyprop- |
| | yl; and X may be |
| | selected from is O, S |
| | or NH. The embodi- |
| | ment shown shows |
| | N in position 3. |
| | However, in other |
| | embodiments N may |
| | be in position 4, 5 or |
| | 6 of the aromatic ring. |

In specific embodiments of the invention, the linker may be able to support multiple fluorescent whitening cores, examples of such linkers are given below, which may be combined in each position with one of the general cores, (I) and (II).

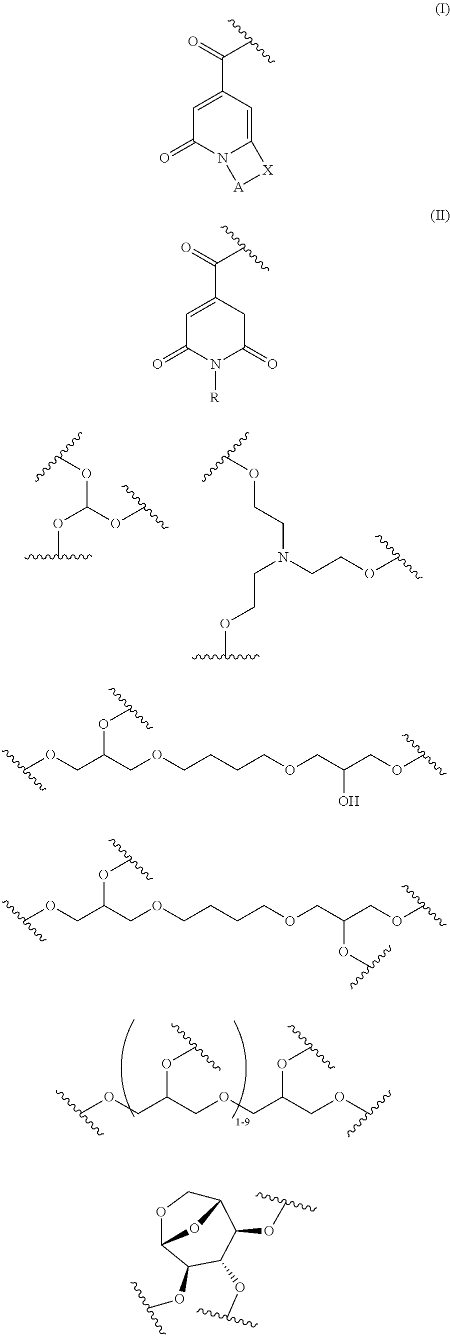

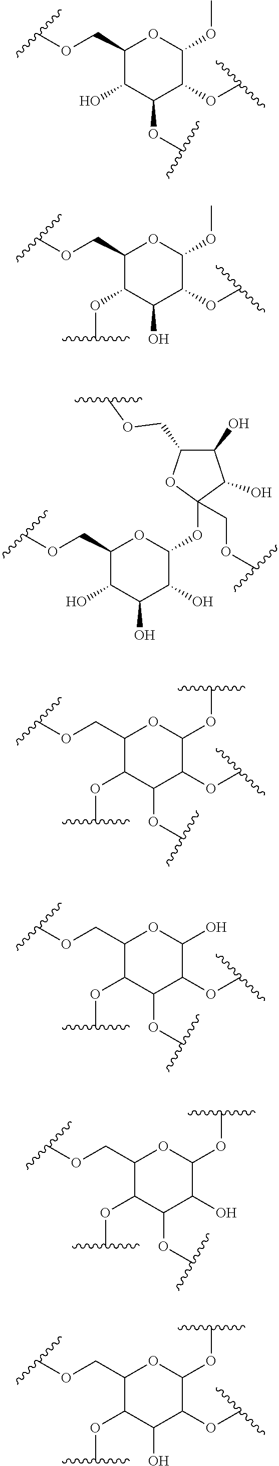

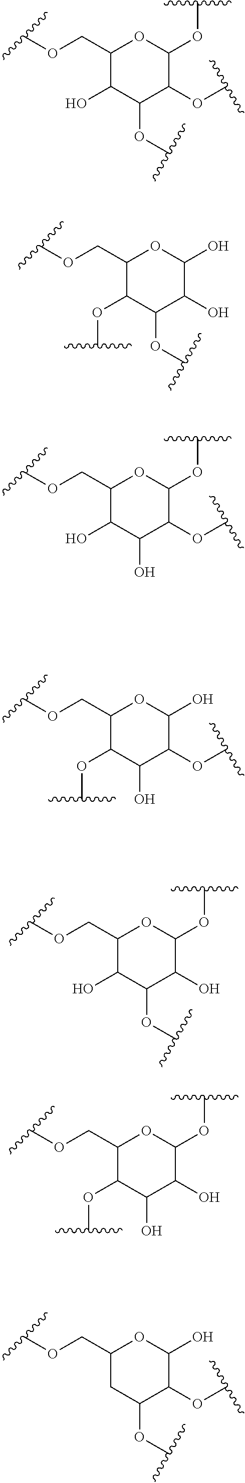

The fluorescent whitening agents or optical brighteners, according to the present invention, have excellent chemical stability at relatively high pH, for example the typical pH of a laundry detergent formulation. They also have very suitable thermal stability in such applications, being preparable (see below) by synthetic methods involving temperatures of up to 130° C.

The fluorescent whitening agents or optical brighteners according to the present invention may be used in a range of applications including, but not limited to, laundry, dishwashing, paper manufacturing, personal care (cosmetics, hair care, etc.), inks, coatings, and adhesives. In these applications, the fluorescent whitening agent or optical brightener is able to make materials, for example cotton, appear whiter, whilst also masking yellowing on aging.

The fluorescent whitening agent or optical brightener may be used in all types of formulation, such as liquids, powders, sheets, or bars, in different ratios and loadings as appropriate.

### Synthesis

The fluorescent whitening agent or optical brightener may be prepared from readily available starting materials, such as citric acid or citrazinic acid. Preparation of the target compounds of the present invention (Scheme 1) is advantageously more environmentally acceptable, as a result of the use of common, and renewable building blocks, such as, for example, citric acid, manufactured in large scale by fermentation.

Each core structure may be readily synthesised by heating citric acid with a second amine building block in the presence of water, with water itself as the major by-product. Water-based synthesis is safer, cleaner, more environmentally friendly and highly scalable.

Further functionalisation of the core units to modify and/or improve substantivity of the resultant fluorescent whitening agent or optical brightener in the desired application may be readily achieved by a second, facile reaction with either an alcohol or amine.

Scheme 1: Representative Synthetic Route Used for Optical Brighteners of the Present Invention, where Y is as Defined in the First Aspect of the Invention, and X¹ is any Suitable Leaving Group

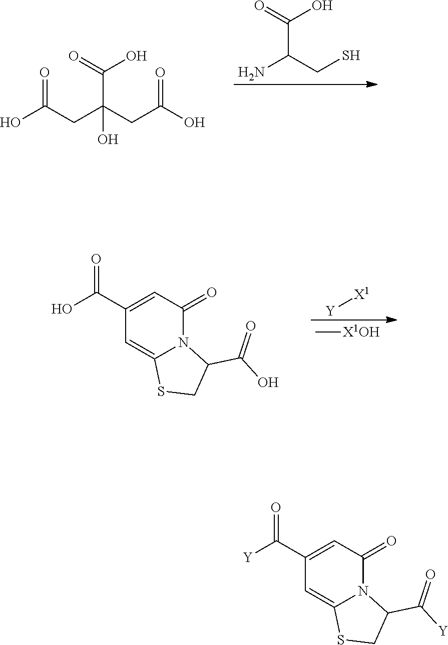

In comparison, preparation of existing optical brighteners, such as benchmarks DAS 1, DAS 2 and FB28 (Scheme 2A), require complex synthetic methods comprising 5 synthetic steps, as well as the use of environmentally undesirable materials, such as precious metal catalysts and hazardous materials such as cyanuric chloride. Similarly, the preparation of the optical brightener DSPB (Scheme 2B) requires complex synthetic steps involving environmentally undesirable and/or hazardous materials, such as trimethyl phosphite and dimethylformamide (DMF). Both families of molecules (Scheme 2A or Scheme 2B) use petrochemical-derived raw materials.

### Scheme 2A: Synthetic Route Used for Current Benchmark Optical Brighteners Such as DAS1, DAS2 and FB28

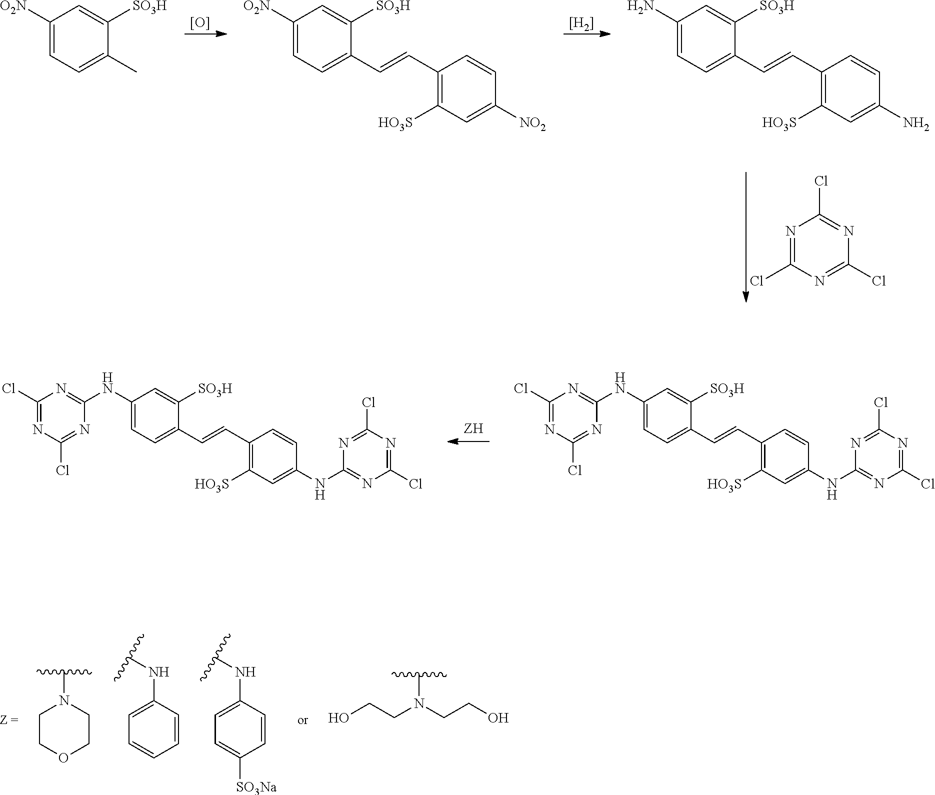

### Scheme 2B: Synthetic Route Used for Current Benchmark Optical Brightener DSPB

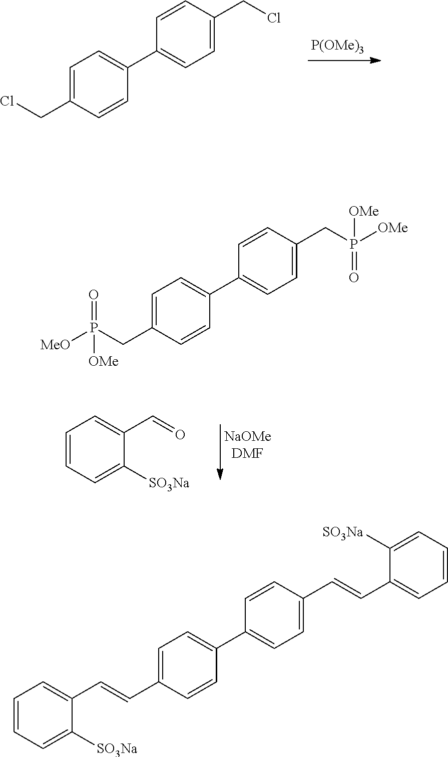

General Synthesis of Fluorescent Whitening Agent Core Units (Bases for Formula (I)) from Citric Acid

A mixture of citric acid (1 eq.) and corresponding amine (1 eq.) were dissolved in water (1 vol). The reaction mixture was heated to 100° C. to allow all water to distil. The reaction temperature was then increased to 140° C. to melt the resulting residues, the molten mixture was then stirred for 16 h and the water of reaction allowed to distil. The resulting resinous material was cooled <100° C. before adding fresh water (1 vol.). The mixture was then cooled to ambient temperature with vigorous stirring to disperse the product before filtration. The isolated solids were washed with fresh water (0.5 vol.) before drying in a vacuum oven to give the final product.

### Synthesis of Fluorescent Whitening Agent Core Unit (1a)

A mixture of citric acid (200 g, 1.05 mol) and cysteamine (80.3 g, 1.05 mol) were dissolved in water (300 ml). The reaction mixture was heated to 100° C. to allow all water to distil. The reaction temperature was then increased to 140° C. to melt the resulting residues with stirring for 4 h. The temperature was then increased to 160° C. and the molten mixture was then stirred for 16 h and the water of reaction allowed to distil. The resulting resinous material was cooled <100° C. before adding fresh water (300 ml). The mixture was then cooled to ambient temperature with vigorous stirring to disperse the product before filtration. The isolated solids were washed with fresh water (150 ml) before drying in a vacuum oven to give the final product as a yellow powder in 64.5% yield (131.4 g, 0.68 mol).

Analytical Data: HPLC purity: 99.7%; LCMS (+ve ion): m/z 198.1

### Synthesis of Fluorescent Whitening Agent Core Unit (3a)

A mixture of citric acid (200 g, 1.05 mol) and cysteine (127.2 g, 1.05 mol) were dissolved in water (325 ml). The reaction mixture was heated to 100° C. to allow all water to distil. The reaction temperature was then increased to 140° C. to melt the resulting residues, the molten mixture was then stirred for 16 h and the water of reaction allowed to distil. The resulting resinous material was cooled <100° C. before adding fresh water (325 ml). The mixture was then cooled to ambient temperature, with vigorous stirring, to disperse the product before filtration. The isolated solids were washed with fresh water (150 ml) before drying in a vacuum oven to give the final product as a yellow powder in 61% yield (154.7 g, 0.64 mol).

Analytical Data: HPLC purity: 99.8%; LCMS (+ve ion): m/z 242.2; 1H NMR (DMSO-d₆, 400=MHz, d): 6.59 (s, 1H), 6.52 (s, 1H), 5.46 (d, 1H), 3.92 (t, 1H), 3.60 ppm (d, 1H); ¹³C{¹H} NMR (DMSO-d₆, 75 MHz, d): 169.2, 165.6, 160.7, 150.2, 142.7, 114.8, 97.9, 62.6 ppm.

### Synthesis of Fluorescent Whitening Agent Core Unit (4a)

A mixture of citric acid (200 g, 1.05 mol) and benzylamine (127.2 g, 1.05 mol) was dissolved in water (325 ml). The reaction mixture was heated to reflux for 16 h. The reaction mixture was then dried under vacuum and grinded to provide the final product as a yellow powder in nearly quantitative yield and good purity.

Esterification of Fluorescent Whitening Agent Core Unit (1a) with Ethanol

A mixture of FWA 1a (5 g, 0.026 mol) and p-toluenesulphonic acid (0.04 g, 0.002 mol) were suspended in industrial methylated spirits (IMS, also called Industrial Denatured Alcohol, IDA, 15 ml) and heated to reflux before stirring for 24 h. The reaction was then allowed to boil dry to remove the water of reaction. Fresh IMS (15 ml) was then added to reaction and the reflux continued for a further 24 h. The reaction mixture was then cooled, and the solvent removed at reduced pressure to give the target ester in near quantitative yield, as a beige powder.

Analytical Data: HPLC purity: 95.8%; LCMS (+ve ion): m/z 226.2

Esterification of Fluorescent Whitening Agent Core Units with Low-Boiling Alcohols (<100° C.)

A mixture of prepared FWA core (1 eq.) was suspended in alcohol (3 vol.) and heated to reflux before stirring for 24 h. The reaction was then allowed to boil dry to remove the water of reaction. Fresh alcohol (3 vol.) was then added to reaction and the reflux continued for a further 24 h. The reaction mixture was then cooled, and the solvent removed at reduced pressure to give the target ester in near quantitative yield.

Esterification of Fluorescent Whitening Agent Core Unit with High-Boiling Alcohols (>100° C.)

A mixture of prepared FWA core (1 eq.) was suspended in alcohol (1.1 eq. per carboxylic acid group) with p-toluenesulphonic acid (0.08 eq.). The resulting mixture was then heated to >100° C., with 130° C. being the preferential reaction temperature, and the water of reaction allowed to distil over 16 h. The excess alcohol was then removed under reduced pressure or azeotropic distillation where appropriate. For alcohols with high boiling points the desired esters were purified either by column chromatography or by precipitation from acetone.

Amidation of Ethyl Ester of FWA Core 1a with Ethylenediamine

A prepared sample of the ethyl ester of FWA core 1a (2.25 g, 0.01 mol) was suspended in Industrial Methylated Spirit (IMS, 15 ml) before adding ethylenediamine (1.17 g, 0.02 mol) to give an instant orange colour. The resulting mixture was heated to reflux and stirred for 16 h. The reaction was then cooled before removing the excess alcohol under reduced pressure to give the desired amide as a beige powder in 82% yield (1.91 g, 0.008 mol).

Analytical Data: HPLC purity: 95.6%; LCMS (+ve ion): m/z 240.2

### Amidation of Functionalised Fluorescent Whitening Agents

A prepared sample of the ethyl ester of the desired FWA core (1 eq.) was suspended in IMS (5 vol.) before adding the target amine (2 eq. per carboxylic acid group) at room temperature. The resulting mixture was heated to reflux and stirred for 16 h. The reaction was then cooled before removing the excess alcohol under reduced pressure to give the desired amide.

### Carriers

In concentrated and granular detergent compositions, the compositions are typically provided to an end user for use as a diluted or dissolved solution. In such a solution, the pH that a fluorescent whitening agent is exposed to, prior to use, can be particularly high, which can give rise to storage issues, in particular with regards to storage stability. There is therefore a need for improved delivery compositions compatible with environmentally acceptable fluorescent whitening agents, particularly those agents defined according to the present invention.

Similarly, fluorescent whitening agents may be incorporated into fabric washing detergent compositions. However, mere incorporation does not guarantee that the fluorescent whitening agent will be able to absorb onto a fabric being washed. One of the functions of a detergent composition is to absorb onto the surfaces of fabric materials and preferentially adhere, such that oils and other residues are removed. Typically, this is achieved using surfactants. Surfactants may be classified as anionic, non-ionic, and cationic. There is therefore a need for a formulation of a fabric washing detergent composition incorporating a fluorescent whitening agent, such that the fluorescent whitening agent can remain absorbed on a fabric material surface after a washing process, particularly where the washing process is followed by a rinsing process. There is therefore a need for a fabric washing detergent composition capable of delivering environmentally acceptable fluorescent whitening agents during a washing process, where they remain in-situ on the material after such a process.

Particularly, there is a need for delivery compositions which enable environmentally acceptable fluorescent whitening agents, specifically those structures defined according to the present application, which provide delayed release of the fluorescent whitening agent at a specific point of the washing process. Specifically, at a point in a washing process where the fluorescent whitening agent has the highest probability of adsorption onto a fabric. Alternatively, rapid release of the fluorescent whitening agent or optical brightener may also be advantageous in certain circumstances, as this would enable the fluorescent whitening agent to adsorb onto one fabric before significant concentrations of the surfactant, for example, are present in a washing method, so as to compete with the fluorescent whitening agent for adsorption onto a fabric.

In an embodiment of the invention, fabric washing detergent compositions are provided. The fabric washing detergent compositions may be in a number of different formats, such as compositions for hand washing, machine washing, uncoloured garments and coloured garments.

Compositions which provide an oxidising agent are widely used and these can also give rise to stability problems for environmentally acceptable fluorescent whitening agents, such as those structures defined in the present application. There is therefore a need for delivery compositions which mitigate or ameliorate issues caused by oxidising agents present in fabric washing detergent compositions.

The aforementioned problems are particularly relevant for environmentally acceptable fluorescent whitening agents due to the chemical complexity of such molecules, the fact that these molecules are present in small amounts and therefore inherently of high surface area to volume in a composition, and that compounds tailored to environmentally acceptable compositions often have labile groups intended to be degraded in order to have improved biodegradability.

Notably, chemical groups designed to improve biodegradability are typically more labile in the presence of an oxidising agent or in high pH conditions, or combinations thereof.

Furthermore, fluorescent whitening agents used in detergent compositions are preferably present at low levels, for example levels as low as 0.01%. Importantly, high concentrations, such as those caused by individual granules, are unfavourable as this can give rise to an uneven distribution. This is particularly exacerbated due to settling of a granular mixture, for example, during transportation. There is therefore a need for delivery compositions which enable an even distribution throughout the composition, along with any other relevant detergent ingredients in the overall detergent composition, particularly a fabric washing detergent composition, and even more particularly a granular, solid fabric washing detergent composition.

Consequently, in another embodiment of the present invention there is provided a solid carrier composition of a fluorescent whitening agent composition comprising a carrier and a fluorescent whitening agent, in accordance with the above.

In such an embodiment, the fluorescent whitening agent or optical brightener may be absorbed into the structure of the carrier. Additionally, the fluorescent whitening agent or optical brightener may be present in the carrier composition at less than 34% by weight.

In another embodiment the fluorescent whitening agent or optical brightener may be present in the carrier composition at between 1% and 34% by weight. In a preferable embodiment, the fluorescent whitening agent or optical brightener may be present between 15 and 30% by weight. Advantageously this appears to result in less dusting and less wicking of any liquid out of the granule, particularly when the fluorescent whitening agent is provided as a liquid.

The fluorescent whitening agents according to the present invention may be provided in solid or liquid form. The fluorescent whitening agents may particularly be in liquid or low melting form, making them difficult to handle whilst incorporating them into products in which they may be present as an adjuvant. Examples of such products may include detergents, such as fabric washing detergents, cosmetics, such as sunscreens, paper manufacture, such as for paper finishing, and a variety of other applications in which fluorescent whitening agents are incorporated. In these applications the fluorescent whitening agents may be present in a small amount, typically less than 1% by weight, sometimes as low as 0.01% by weight.

In these applications, it is therefore more difficult to handle and incorporate such materials. To overcome this challenge, it is therefore advantageous to increase the weight and volume of the fluorescent whitening agent by incorporating it into a carrier. However, it is important that, particularly when the fabric whitening agent is a liquid or a low melting solid, that it is fully incorporated into the carrier. In particular embodiments of the invention when such a level of incorporation is 34 wt. % or less, the fluorescent whitening agent or optical brightener may be advantageously incorporated into interstices of an otherwise porous structure.

In embodiments of the present invention, the carrier may be selected from one or more of sodium carbonate, potassium carbonate, calcium carbonate, magnesium carbonate cellulose, carboxymethyl cellulose, polyvinyl alcohol of 100,000 or more repeat units, sodium silicate, polyvinylpyrrolidone of 50,000 or more repeat units, or a zeolite. Particularly, such materials are compatible with detergent compositions.

In preferred embodiments of the present invention, the carrier may be a water-soluble inorganic salt. These salts have been found to more rapidly disperse fluorescent whitening agent, particularly when incorporated into aqueous media.

In the present invention, the carrier composition may be prepared by drying the fluorescent whitening agent and the carrier from a solution, as this enables a homogeneous mixture to be produced before the carrier composition is prepared in solid form by drying.

In the present invention, the drying of the composition is preferably achieved using spray drying. Spray drying has been found to enable particularly relatively low solubility fluorescent whitening agents to be effectively combined with soluble components in the form of a carrier, in a substantially homogeneous manner. This is particularly useful where the fluorescent whitening agent is a liquid or a low melting solid, as a homogeneous mixture with a solubilised carrier may be produced for spray drying providing a resultant ‘dry’ and low-dusting granule.

In the present invention, the carrier composition may be prepared by drying of the composition by a fluidised bed. Advantageously drying in this manner is effective where the fluorescent whitening agents are sensitive to high drying temperatures. This is particularly relevant as the fluorescent whitening agents according to the invention are readily biodegradable and often have labile groups such as esters, which makes them temperature sensitive relative to conventional fluorescent whitening agents.

In the present invention, where the carrier composition comprises an insoluble carrier and a low solubility or solid fluorescent whitening agent, it may be prepared by co-granulation of the carrier and the fluorescent whitening agent with a binder. This process avoids high temperatures and is therefore advantageous given the aforementioned problems regarding the stability of the fluorescent whitening agents. The binder may be selected from sodium silicate, or an organic polymer. In most preferred embodiments the binder may be an organic polymer in particular a polyvinyl alcohol or a polyvinyl alcohol copolymer. The polar nature of these polymers advantageously provides more homogeneous compositions than those polymers with low polarities or hydrophilicities.

Polycarboxylic acid may also be an example of a suitable binder material, especially where there is an amine or amide functionality in the fluorescent whitening agent as it provides improved solubilisation of the fluorescent whitening agent during preparation of the carrier composition.

Clearly there is a synergistic effect between proteolytic enzymes and fluorescent whitening agents, and therefore in some further embodiments of the present invention, the carrier composition may further comprise a proteolytic enzyme. This is particularly the case where the fluorescent whitening agent comprises an amide functionality. Additionally, there is synergistic effect between lipolytic enzymes and fluorescent whitening agents, and therefore the carrier composition may further comprise a lipolytic enzyme. This is particularly the case where the fluorescent whitening agent comprises an ester functionality.

The fluorescent whitening agent or optical brightener may be a liquid at ambient temperature.

The fluorescent whitening agent or optical brightener may have a solubility of less than 1 g a litre in water at ambient temperature.

The carrier may be in the form of a polymer sheet, which may form a sachet or pouch for a detergent composition.

### Detergents

According to a further aspect of the present invention there is provided a detergent composition comprising the aforesaid fluorescent whitening agent or optical brightener.

The detergent composition may comprise a detersive surfactant, for example an anionic, cationic, zwitterionic or non-ionic surfactant.

In preferred embodiments the detergent composition comprises non-ionic surfactants.

Advantageously, it has been found that non-ionic surfactants provide a preferred base detergent composition for the deposition of fluorescent whitening agents or optical brighteners of the present invention.

In embodiments of the present invention where the detersive surfactant is a non-ionic surfactant, it may be selected from one or more of a rhamnolipid or sophorolipid. Commercial examples of such surfactants include, but may not be limited to, BioLoop 56L, BioLoop 56L-PG, BioLoop 68L, BioLoop 68L-PG, BioLoop 84L and BioLoop 84L-PG.

In the present invention the fluorescent whitening agents or optical brighteners preferably have an amide functionality.

It has been found that such fluorescent whitening agents or optical brighteners provide better adsorption onto surfaces being cleaned by the detersive composition. Not wishing to be bound by theory is understood that some degree of positive charge on the amide can interact with negatively charged services. It is conventionally understood to be the case when articles are being washed.

It has been found that the present invention can provide improved FWA or optical brightener deposition when used in relevant detergent compositions. It is hypothesised that the FWA or optical brightener co-adsorbs with the cationic surfactant onto surfaces being washed.

In compositions utilising a positively charged surfactant, such as a cationic or zwitterionic surfactant the FWA or optical brightener preferably has Y as OH or OM, M being Na or K.

It has been found that these FWAs or optical brighteners provide improved deposition, possibly because of the combination of the negatively charged FWA or optical brightener and the positively charged surfactant, which together provide improved surface adsorption. This is particularly beneficial for detersive compositions such as fabric softeners.

In the present invention the in-use pH of the detergent composition is preferably in the range from pH 8 to 10. A pH in this range is particularly beneficial for adsorption of ionisable FWA or optical brightener.

In the present invention the detergent composition is preferably a laundry detergent composition, such as includes fabric softeners, whitening agents, pre-treatment agents as well as conventional solid, liquid, gel, pouch detergent compositions, as well as heavy duty, light duty ‘whites’ and ‘colours’ detergent compositions.

In the present invention the detergent composition may preferably be a fabric washing detergent composition selected from one or more of a laundry detergent, a laundry fabric softener, a bleach, or a booster laundry aid (such as whitening agent).

In the present invention the detergent composition preferably further comprises a proteolytic enzyme and the FWA or optical brightener comprises an amide linkage.

The detergent composition of the present invention may further comprise a lipolytic enzyme, especially in embodiments in the FWA or optical brightener comprises an ester linkage.

The detergent composition of the present invention may be in the physical form of the detergent composition is a powder (granulate), liquid, a gel, a pouch, a tablets, or a solid sheet.

Preferably the physical form is solid. This is particularly the case for FWA or optical brightener where m is 3 or more and L is a polymer. Such embodiments provide an FWA or optical brightener in a solid form which is compatible with other solids and not subject to hydrolysis in the undiluted state. When the solid is diluted, such as during washing, the detergent composition components, such as enzymes or pH agents can trigger hydrolysis, which releases the FWA or optical brightener into the washing composition.

Included within the scope of the invention are detergent compositions selected from one or more of a laundry detergent, a laundry fabric softener, a bleach, or a booster laundry aid (such as whitening agent).

The present invention therefore further provides a fluorescent whitening agent or optical brightener solid carrier composition comprising a carrier and a fluorescent whitening agent or optical brightener in accordance with the foregoing description.

The compounds according to the present invention may be capable of providing a fluorescent whitening or optical brightening effect, as well as being readily, ultimately, or inherently biodegradable. The compounds may all have a common renewable building block, which makes their production environmentally friendly. Additionally, the compounds may have one or more of the characteristics of: being substantive to fabric, such as natural materials including cotton and cellulose; being compatible with a synthetic fabric materials; providing peak fluorescent output in the blue (450 and 495 nm) or green (495-570 nm) portions of the visible spectrum; providing high fluorescence in solid form and solution; providing high fluorescence when absorbed on a fabric and solution; and/or providing higher absorption onto cotton than 2,2′-Stilbenedisulphonic acid.

Other applications of the fluorescent whitening agents as defined in the present invention:

### Fabrics

FWAs or optical brighteners of the present invention may be used in fabric manufacturing.

FWAs or optical brighteners of the present invention may be used in finishing processes for cotton. Preferably in the treatment of griege (loom-state) cloth into finished fabric.

FWAs or optical brighteners of the present invention may be used in melt spinning thing synthetic fibres.

### Paper

FWAs or optical brighteners of the present invention may be used in paper manufacturing. Preferably during “wet” manufacturing processes.

FWAs or optical brighteners of the present invention may be used in paper finishing.

FWAs or optical brighteners of the present invention may be used to treat cellulose fibre before paper manufacture.

### Cosmetics

FWAs or optical brighteners of the present invention may be used in conjunction with emollient to provide cosmetics compositions.

FWAs or optical brighteners of the present invention may be used to produce sunscreen compositions.

The advantages of FWA or optical brightener cosmetics is that they provide an increase in the apparent lustre, shine or colour intensity of the cosmetics and do so in an environmentally acceptable format.

### EXAMPLES

### Example 1—Evaluation of Fluorescent Whitening Agents

Fluorescent whitening agents and optical brighteners of the present invention have been evaluated with respect to chemical and UV stability, quantum yield, fluorescence/whiteness effect, biodegradability, and substantivity onto cellulose as compared to commercial benchmarks.

### Relative Quantum Yield

A solution of FWA was prepared in 0.1M sodium carbonate. The solution was diluted down till a UV absorbance of 0.1 was measured (Agilent™ Cary UV-vis), this was then diluted further to give a range of UV absorbance readings. These solutions were then run to measure the fluorescence response (Agilent™ Cary Eclipse fluorescence spectrometer). The integrated area from the fluorescence measurements was plotted against the UV absorbance readings. This was compared to the gradient measure for a quinine sulphate in 0.1M sulfuric acid standard to calculate the relative quantum yield. The results are provided below in Tables 1 and 2.

TABLE 1
Relative quantum yield of benchmark FWA or optical brightener

| | Emission | Relative |
|---|---|---|
| | Maximum | Quantum |
| Structure | (nm) | Yield |
| 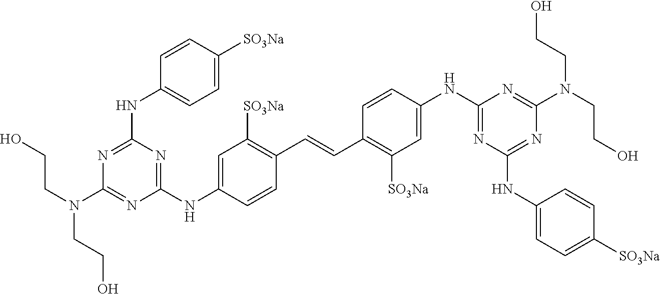 | 430.9 | 33% |
| Benchmark 2 (DAS-2) | | |
| CAS 16470-24-9 | | |
| 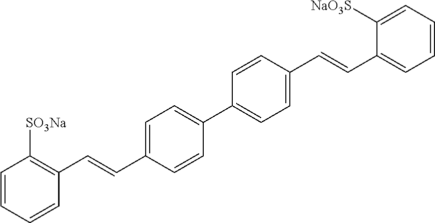 | 429.8 | 78% |
| Benchmark 4 (DSBP) | | |
| CAS 27344-41-8 | | |

TABLE 2
Relative quantum yield of FWA or optical brightener examples

| | Emission | Relative |
|---|---|---|
| | Maximum | Quantum |
| Structure | (nm) | Yield |
| 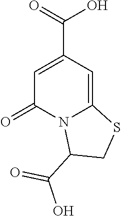 [SMILES: O=C(O)c1cc2n(c(=O)c1)C(C(=O)O)CS2] | 417.0 | 72% |
| 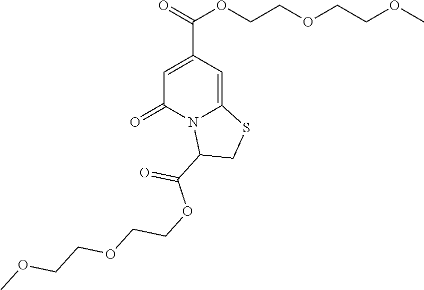 | 419.8 | 77% |
| 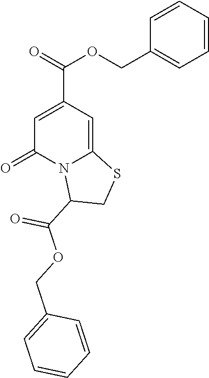 [SMILES: O=C(OCc1ccccc1)c1cc2n(c(=O)c1)C(C(=O)OCc1ccccc1)CS2] | 418.9 | 45% |
| 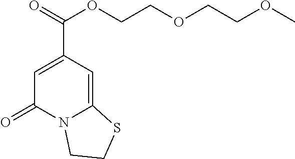 [SMILES: COCCOCCOC(=O)c1cc2n(c(=O)c1)CCS2] | 417.8 | 71% |
| 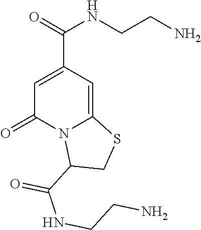 [SMILES: NCCNC(=O)c1cc2n(c(=O)c1)C(C(=O)NCCN)CS2] | 428.9 | 59% |
| 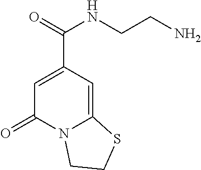 [SMILES: NCCNC(=O)c1cc2n(c(=O)c1)CCS2] | 423.8 | 59% |
| 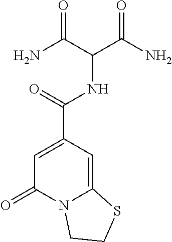 [SMILES: NC(=O)C(NC(=O)c1cc2n(c(=O)c1)CCS2)C(N)=O] | 432.0 | 60% |

The relative quantum yields of compounds according to the invention can readily be seen to be comparable with, or better than, the prior art standards.

### Fluorescence/Whiteness Effect as Measured on Cellulose Powder:

Colour measurement was made with a Lovibond® LC100 spectrocolorimeter. The surface colour of the powder was quantified using a series of values L*, a*, and b* from the colour model CIELAB defined by the International Commission on Illumination's (Commission Internationale de l'Eclairage). L* is a measure of the amount of white or black in a sample; higher L* values indicate a lighter coloured sample. A measure of the amount of red or green in a sample was determined by “a*” values. A measure of the amount of blue or yellow in a sample was determined by “b*” values; lower (more negative) b* values indicate more blue on a sample. Colour can also be measured using a different model, CIE L*C*h*, where C* represents chroma, and h* represents the hue angle. The powder from each substantivity test was measured against blanked washed cellulose and set the start point for the next UV stability test. The results are provided below in Table 3.

TABLE 3
Results for example compounds

| | | | | | h* (0 = red |
|---|---|---|---|---|---|
| | | a* | b* | | 90 = yellow |
| | | (−ve green | (−ve blue | | 180 = green |
| Sample | L* | +ve red) | +ve yellow) | C* | 270 = blue) |
| Blank | 94.8 | 0.6 | 2.2 | 2.3 | 75.4 |
| 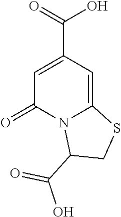 [SMILES: O=C(O)c1cc2n(c(=O)c1)C(C(=O)O)CS2] | 97.6 | 0.2 | 2.3 | 2.3 | 83.9 |
| 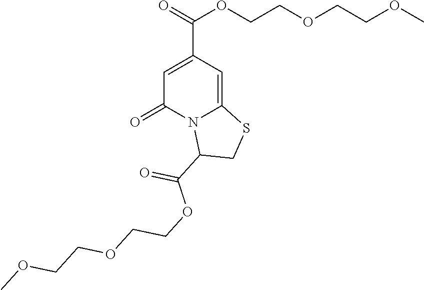 | 98.4 | 0.1 | 2.2 | 2.2 | 86.6 |
| 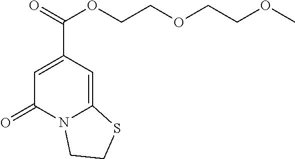 [SMILES: COCCOCCOC(=O)c1cc2n(c(=O)c1)CCS2] | 97.7 | 0.4 | 1.8 | 1.9 | 77.4 |
| 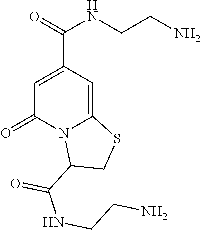 [SMILES: NCCNC(=O)c1cc2n(c(=O)c1)C(C(=O)NCCN)CS2] | 97.7 | 0.2 | 2.5 | 2.5 | 84.6 |
| 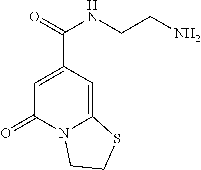 [SMILES: NCCNC(=O)c1cc2n(c(=O)c1)CCS2] | 99.6 | −0.3 | 0.9 | 1 | 109.6 |
| 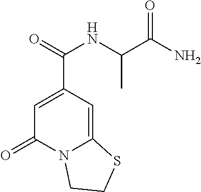 [SMILES: CC(NC(=O)c1cc2n(c(=O)c1)CCS2)C(N)=O] | 98.4 | 0.1 | 1.6 | 1.6 | 87.7 |
| 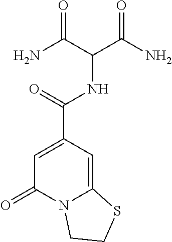 [SMILES: NC(=O)C(NC(=O)c1cc2n(c(=O)c1)CCS2)C(N)=O] | 98.8 | 0.2 | 1.1 | 1.1 | 80.8 |

All compounds show a substantial shift towards a blue hue (h*=270) compared to the blank, demonstrating efficacy in terms of both substantivity and optical effect.

UV Stability (i.e. Light Fastness)

Cellulose powder as prepared above was exposed to UV in a UV box under a 4 W lamp (365 nm) for a period of 3 months. The samples were then visually inspected, picture taken, and colour measurement taken again to monitor loss in whiteness and changes from blue to yellow or darker tone. The results are provided below in Table 4.

TABLE 4
Results for example compounds after exposure to UV

| | | | | | h* (0 = red |
|---|---|---|---|---|---|
| | | a* | b* | | 90 = yellow |
| | | (−ve green | (−ve blue | | 180 = green |
| Sample | L* | +ve red) | +ve yellow) | C* | 270 = blue) |
| 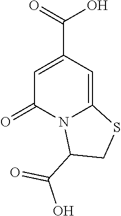 [SMILES: O=C(O)c1cc2n(c(=O)c1)C(C(=O)O)CS2] | 99.6 | 0.2 | 2.6 | 2.7 | 85.6 |
| 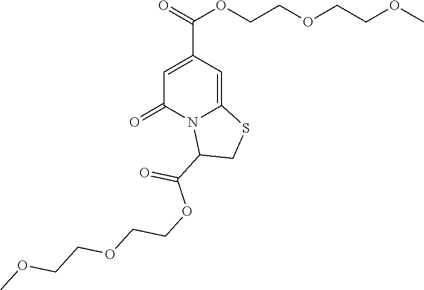 | 97.4 | 0.7 | 4 | 4 | 80.1 |
| 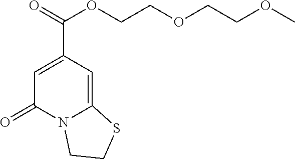 [SMILES: COCCOCCOC(=O)c1cc2n(c(=O)c1)CCS2] | 95.6 | 0.6 | 2.4 | 2.4 | 75 |
| 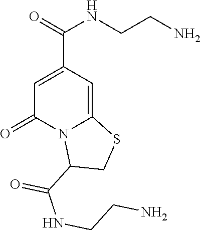 [SMILES: NCCNC(=O)c1cc2n(c(=O)c1)C(C(=O)NCCN)CS2] | 97.6 | 0.2 | 3.2 | 3.2 | 85.8 |
| 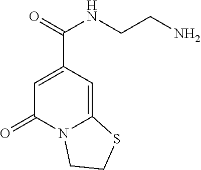 [SMILES: NCCNC(=O)c1cc2n(c(=O)c1)CCS2] | 97.4 | 0.4 | 2.3 | 2.3 | 79.3 |
| 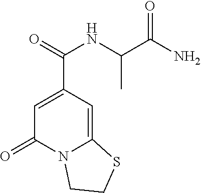 [SMILES: CC(NC(=O)c1cc2n(c(=O)c1)CCS2)C(N)=O] | 97.3 | 0.3 | 3.1 | 3.1 | 85.3 |
| 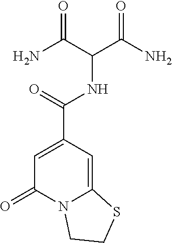 [SMILES: NC(=O)C(NC(=O)c1cc2n(c(=O)c1)CCS2)C(N)=O] | 98.2 | 0.2 | 2.1 | 2.1 | 84.1 |

The FWAs of the present invention are stable under UV under the test conditions.

### Chemical Stability

FWA or optical brightener was weighed at 0.0100-0.0200 g into a 100 mL volumetric flask and made up to volume using deionised water. The solution was sonicated for 5 minutes to ensure a uniform dispersion has been created. The solution was split into five 10 ml samples. Sample 1 was a blank with 0.2 mL of DI water added. To sample 2 0.1 mL of 1M Sodium hydroxide solution and 0.1 mL of DI water were added to sample 3 0.1 mL of 1M Sodium bicarbonate solution and 0.1 mL of DI water were added, to sample 4 0.1 mL of 1M Sodium hydroxide solution and 0.1 mL of 0.75M Hydrogen peroxide were added, to sample 5 0.1 mL of 1M Sodium carbonate solution and 0.1 ml of 0.75M Hydrogen peroxide were added. All samples were heated to 40° C. for 48 h and then analysed by HPLC. Blank sample is used to identify the FWA or optical brightener retention time and maximum response. Any hydrolysis and oxidation of the FWA or optical brightener would result in new compound(s) appearing on the chromatogram at different retention time together with a reduction of the FWA or optical brightener response. The other samples are reported as FWA or optical brightener % response vs blank. Conditions 3 (Carbonate) and 5 (Carbonate and Hydrogen peroxide) are the most relevant to laundry applications (e.g. heavy-duty powder detergent). The results are provided below in Table 5.

TABLE 5
Chemical stability of example compounds

| | Condition 3 | Condition 5 | |
|---|---|---|---|
| | (% response | (% response | |
| Structures | vs blank) | vs blank) | Comment |
| 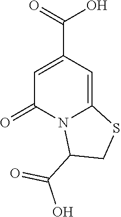 [SMILES: O=C(O)c1cc2n(c(=O)c1)C(C(=O)O)CS2] | 100 | 99.8 | Excellent stability |
| 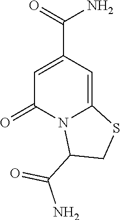 [SMILES: NC(=O)c1cc2n(c(=O)c1)C(C(N)=O)CS2] | 86 | 86.7 | Good stability, limited partial saponification of one of the amide groups. |

The monitoring of chemical stability of the FWAs shown in Table 5 confirms excellent stability of unfunctionalized structures and those functionalised with amines in the presence of high pH and oxidants. These results show a high degree of compatibility with laundry applications.

The fluorescent whitening agents of the present invention have been further evaluated for biodegradability, and substantivity onto cellulose as compared to commercial benchmarks.

### Substantivity on to Cellulose:

A buffer solution was prepared by weighing 0.10 g SDS, 1.0 g glycerol, 8.4 g sodium carbonate, 1.7 g sodium bicarbonate and 0.4 g ethylene diamine tetra acetic acid (EDTA) into a 50 ml beaker and transferring it into a 2000 ml beaker. The 50 ml beaker was rinsed into the 2000 ml beaker using tap water and made to around 1 L using tap water while stirring until fully dissolved. This solution was transferred into a 2 L volumetric flask and made up to volume using tap water.

FWA was weighed at 0.0100-0.0200 g into a 100 mL volumetric flask and made up to volume using buffer solution. The solution was sonicated for 5 minutes to ensure a uniform dispersion has been created.

Into a 250 ml beaker, 10 g of Thermo Scientific™ microcrystalline cellulose 50 μm powder and 50±1 g of FWA solution were added and their exact weight was recorded. The cellulose/FWA slurry was stirred for 20 minutes before being filtered in a filtration unit with a polypropylene filter cloth. The filtrate (mother liquor) was collected. The cellulose was rinsed three times with 50 ml of tap water, the cellulose was re-slurred by simple stirring before being filtered. Combined filtrates (mother liquor and rinses 1 to 3) were collected and weighted. The cellulose cake was removed from the filtration unit and dried in a vacuum oven at 35° C. for further visual, fluorescence and light fastness tests.

To measure the quantity of FWA retained on cellulose, the initial sample and combined filtrates were run by HPLC. Using the peak areas for the FWA and input-output weights, the retained amount of FWA can be calculated. The results are provided below in Tables 6 and 7.

Substantivity can be defined by the following criteria: poor (<25%), moderate (25-50%), good (50-85%), and excellent (>85%).

TABLE 6
Substantivity of compounds onto cellulose of benchmark compounds

| | | | Substantivity |
|---|---|---|---|
| | | Substan- | (excellent, |
| | | tivity | good, |
| | | (% on | moderate, |
| No | Structures | cellulose) | poor) |
| 1 | 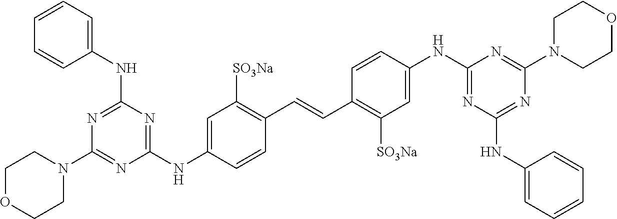 | 98% | Excellent |
| | Benchmark 1 (DAS-1) | | |
| | CAS 16090-02-1 | | |
| 2 | 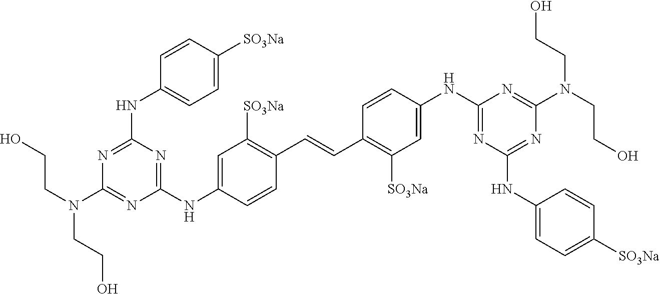 | 98% | Excellent |
| | Benchmark 2 (DAS-2) | | |
| | CAS 16470-24-9 | | |
| 3 | 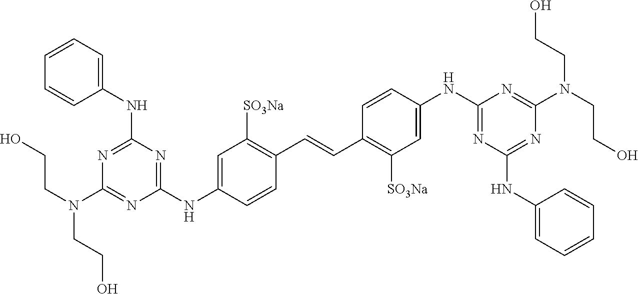 | 98% | Excellent |
| | Benchmark 3 (FB28) | | |
| | CAS 4193-55-9 | | |
| 4 | 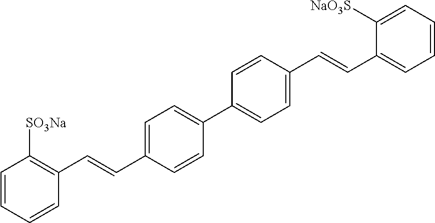 | 93% | Excellent |
| | Benchmark 4 (DSBP) | | |
| | CAS 27344-41-8 | | |

TABLE 7
Substantivity of compounds onto cellulose of example compounds

| | Substantivity | Substantivity |
|---|---|---|
| | (% on | (excellent, good, |
| Structures | cellulose) | moderate, poor) |
| 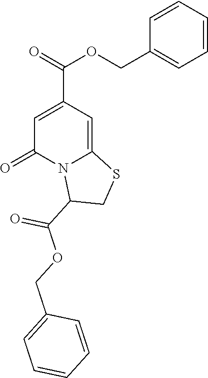 [SMILES: O=C(OCc1ccccc1)c1cc2n(c(=O)c1)C(C(=O)OCc1ccccc1)CS2] | 97% | Excellent |
| 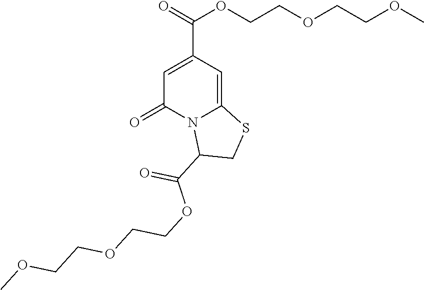 | 75% | Good |
| 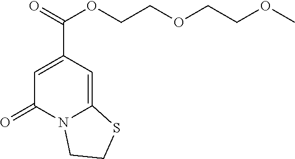 [SMILES: COCCOCCOC(=O)c1cc2n(c(=O)c1)CCS2] | 77% | Good |
| 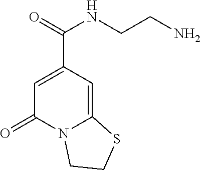 [SMILES: NCCNC(=O)c1cc2n(c(=O)c1)CCS2] | 52% | Good |
| 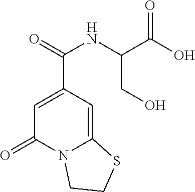 [SMILES: O=C(NC(CO)C(=O)O)c1cc2n(c(=O)c1)CCS2] | 61% | Good |
| 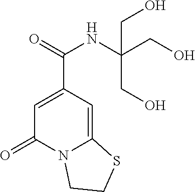 [SMILES: O=C(NC(CO)(CO)CO)c1cc2n(c(=O)c1)CCS2] | 34% | Moderate |
| 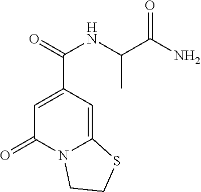 [SMILES: CC(NC(=O)c1cc2n(c(=O)c1)CCS2)C(N)=O] | 27% | Moderate |

Compounds shown in Table 7 show levels of performance (with regards to substantivity) approaching comparability with the industry standards shown in Table 6, whilst also being non-toxic. Additionally, these compounds are biodegradable, non-bio-accumulative and producible from sustainable materials.

Substantivity onto Fabric:

10 L of tap water was added to a top-loading washing machine (capacity 4.5 kg) at 35° C. Standard swatches of fabric (10 cm×10 cm, supplier: CFT, Center for Testmaterials B.V.) were added to the water, along with 20 g of ECE-2 dye transfer test detergent ISO 105 C-08 and 0.1 g of FWA. The washing machine was run for 9 minutes and then allowed to fully drain.

5 L of cold water was added to rinse the fabric and the washing machine was run for 3 minutes before being drained, and then the fabric was subject to a second rinse with 5 L of cold water in a 3-minute run. After rinsing, the fabric swatches were spin dried for 3 minutes before being fully air dried.

The surface colour of the swatches was measured in duplicate with a Lovibond® LC100 spectrocolorimeter. Average values for the surface colour are provided below in Table 8.

TABLE 8
Substantivity of compounds onto fabric for example compounds

| | h* (0 = red 90 = yellow 180 = green 270 = blue) | | |
|---|---|---|---|
| | PCN01 | CN17 (cotton, | |
| | (polyester/ | cretonne, | CN11 (cotton, |
| | cotton | bleached, | cretonne, |
| | 65/35, woven, | mercerized, | bleached, |
| Structures | 180 g/m²) | 155 g/m²) | 155 g/m²) |
| Blank | 165.9 | 128.8 | 152.3 |
| 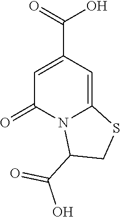 [SMILES: O=C(O)c1cc2n(c(=O)c1)C(C(=O)O)CS2] | 192.4 | 209.9 | 188.3 |
| 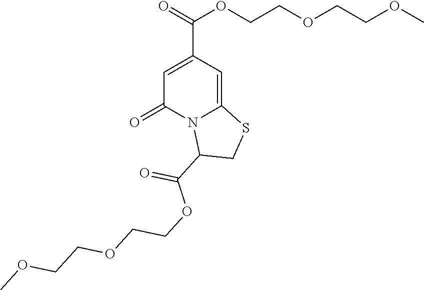 | 195.5 | 160.9 | 195.2 |
| 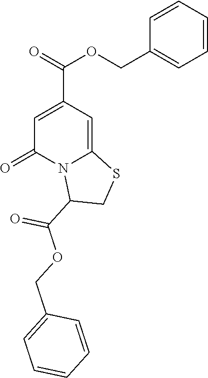 [SMILES: O=C(OCc1ccccc1)c1cc2n(c(=O)c1)C(C(=O)OCc1ccccc1)CS2] | 228.3 | 176.5 | 182.9 |
| 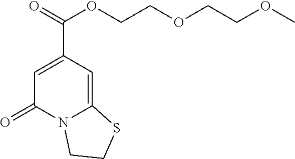 [SMILES: COCCOCCOC(=O)c1cc2n(c(=O)c1)CCS2] | 175.1 | 188.8 | 158.6 |
| 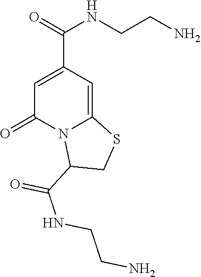 [SMILES: NCCNC(=O)c1cc2n(c(=O)c1)C(C(=O)NCCN)CS2] | 194.4 | 197.3 | 217.1 |
| 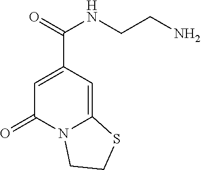 [SMILES: NCCNC(=O)c1cc2n(c(=O)c1)CCS2] | 217.8 | 230.6 | 202.2 |
| 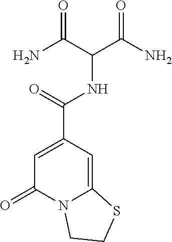 [SMILES: NC(=O)C(NC(=O)c1cc2n(c(=O)c1)CCS2)C(N)=O] | 198.5 | 198.3 | 214.0 |

All compounds show a substantial shift towards a blue hue (h*=270) compared to the blank, demonstrating efficacy in terms of both substantivity and optical effect.

### Example 2—Biodegradability Assessment Using Biowin™

The chemical structures of the present invention are biodegradable.

For the purposes of the present invention:

An inherently biodegradable fluorescent whitening agent is defined as having >20% but <60% biodegradability in water as measured by OECD 301A-F testing.

A readily biodegradable fluorescent whitening agent is defined as having the ability to biodegrade quickly and completely in water (either ≥70% dissolved organic carbon DOC removal, ≥60% theoretical carbon dioxide or ≥60% theoretical oxygen demand, depending on OECD 301A-F test methods) in a 10-day window within 28 days.

Biowin™ software was used to provide predictions of biodegradability. This method of prediction of biodegradability is available as part of the Estimation Program Interface (EPI) Suite™ of software provided by the United States Environmental Protection Agency (EPA). Environ. Sci. Technol., 1994, 28, 459-465, shows an example of the group contribution method for predicting probability and the rate of aerobic biodegradation. This is the type of method used by Biowin™.

Biowin 3 and Biowin 5 models are of particular relevance to the present invention. The predicted biodegradability of some of the compounds according to the present invention were assessed using these models. If the Biowin 3 (ultimate survey model) result is >=2.75 (i.e. “weeks” or faster) and the Biowin 5 (MITI linear model) probability is >=0.5, then the prediction is YES (readily biodegradable). If this condition is not satisfied, the prediction is NO (not readily biodegradable). The results are provided below in Tables 9 and 10:

TABLE 9
Biowin predicted biodegradability score for the benchmark compounds

| | Biowin | Biowin | Interpretation of Biowin 3 & 5 |
|---|---|---|---|
| Benchmark structures | 3 | 5 | prediction. |
| 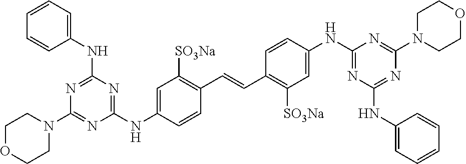 | 0.0223 | −2.1266 | Very poor based on Biowin criteria. Confirmed experimentally as “neither readily nor inherently biodegradable”, see ECHA (https://echa.europa.eu/registration- dossier/-/registered- dossier/14744/5/3/1) |
| Benchmark 1 (DAS-1) | | | |
| CAS 16090-02-1 | | | |
| 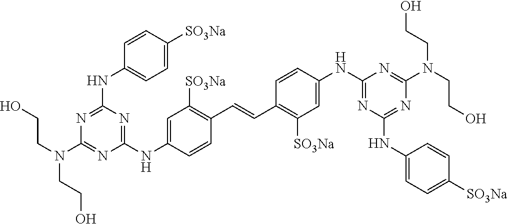 | 0.8228 | −1.3985 | Very poor, fails both Biowin 3 & Biowin 5 criteria for readily biodegradability by a large margin. Confirmed experimentally as “neither readily nor inherently biodegradable”, see ECHA (https://echa.europa.eu/registration- dossier/-/registered- dossier/16029/5/3/2) |
| Benchmark 2 (DAS-2) | | | |
| CAS 16470-24-9 | | | |
| 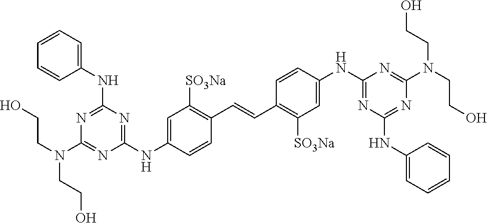 | 0.5999 | −1.3547 | Very poor, fails both Biowin 3 & Biowin 5 criteria for readily biodegradability by a large margin. Confirmed experimentally as “neither readily nor inherently biodegradable”, see ECHA (https://echa.europa.eu/registration- dossier/-/registered- dossier/15216/5/3/2) |
| Benchmark 3 (FB28) | | | |
| CAS 4193-55-9 | | | |
| 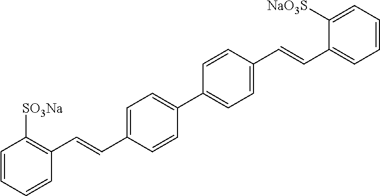 | 2.3375 | −0.6302 | Poor to average, fails both Biowin 3 & Biowin 5 criteria for readily biodegradability. Yet significantly better score than above other benchmark. Confirmed experimentally as “moderately removed from water” (0% DOC removal in 28 days with OECD 301A method and 43.7% DOC removal with OECD 302B method), see ECHA (https://echa.europa.eu/registration- dossier/-/registered- dossier/13511/5/3/2) |
| Benchmark 4 (DSBP) | | | |
| CAS 27344-41-8 | | | |

TABLE 10
Biowin predicted biodegradability score for structures within this invention

| | Biowin | Biowin | |
|---|---|---|---|
| Structures | 3 | 5 | Interpretation of Biowin 3 & 5 prediction. |
| 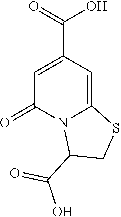 [SMILES: O=C(O)c1cc2n(c(=O)c1)C(C(=O)O)CS2] | 3.3411 | 0.5280 | Readily Biodegradable according to Biowin criteria |
| 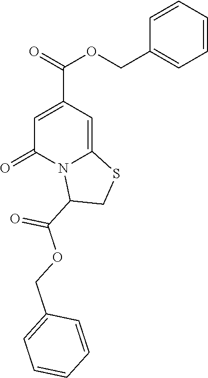 [SMILES: O=C(OCc1ccccc1)c1cc2n(c(=O)c1)C(C(=O)OCc1ccccc1)CS2] | 2.5380 | 0.2877 | Expected to be moderately biodegradable according to Biowin criteria: Biowin 3 score is within 2.25-2.75 range (weeks to months), Biowin 5 score is significantly better than any of the benchmarks. |
| 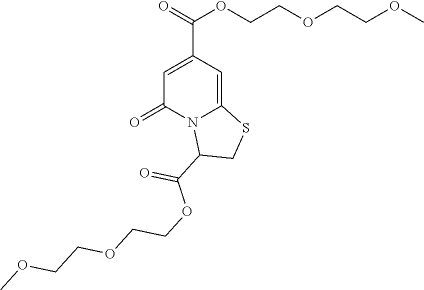 | 2.4062 | 0.6475 | Expected to be biodegradable according to Biowin criteria: Biowin 3 score is within 2.25-2.75 range (weeks to months), Biowin 5 score is above threshold. |
| 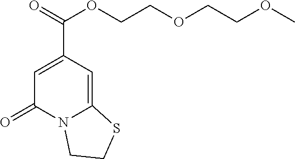 [SMILES: COCCOCCOC(=O)c1cc2n(c(=O)c1)CCS2] | 2.6063 | 0.5448 | Expected to be biodegradable according to Biowin criteria: Biowin 3 score is within 2.25-2.75 range (weeks to months), Biowin 5 score is above threshold. |
| 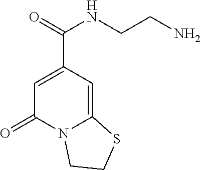 [SMILES: NCCNC(=O)c1cc2n(c(=O)c1)CCS2] | 2.5864 | 0.4374 | Expected to be biodegradable according to Biowin criteria: Biowin 3 score is within 2.25-2.75 range (weeks to months), Biowin 5 score is close to threshold and significantly higher than any of the benchmarks. |
| 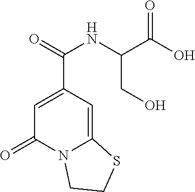 [SMILES: O=C(NC(CO)C(=O)O)c1cc2n(c(=O)c1)CCS2] | 2.9871 | 0.5125 | Readily Biodegradable according to Biowin criteria. |
| 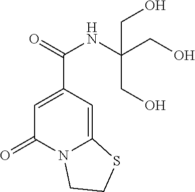 [SMILES: O=C(NC(CO)(CO)CO)c1cc2n(c(=O)c1)CCS2] | 2.6948 | 0.8230 | Readily Biodegradable according to Biowin criteria. |
| 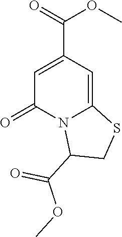 [SMILES: COC(=O)c1cc2n(c(=O)c1)C(C(=O)OC)CS2] | 2.8303 | 0.7135 | Readily Biodegradable according to Biowin criteria. |
| 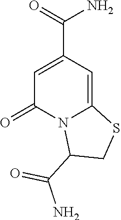 [SMILES: NC(=O)c1cc2n(c(=O)c1)C(C(N)=O)CS2] | 2.5078 | 0.2767 | Expected to be biodegradable according to Biowin criteria: Biowin 3 score is within 2.25-2.75 range (weeks to months), Biowin 5 score is close to threshold and significantly higher than any of the benchmarks. |
| 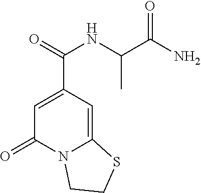 [SMILES: CC(NC(=O)c1cc2n(c(=O)c1)CCS2)C(N)=O] | 2.4458 | 0.2984 | Expected to be biodegradable according to Biowin criteria: Biowin 3 score is within 2.25-2.75 range (weeks to months), Biowin 5 score is close to threshold and significantly higher than any of the benchmarks. |
| 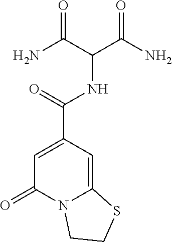 [SMILES: NC(=O)C(NC(=O)c1cc2n(c(=O)c1)CCS2)C(N)=O] | 2.3275 | 0.3383 | Expected to be biodegradable Biowin 3 score is within 2.25-2.75 range (weeks to months), Biowin 5 score is close to threshold and significantly higher than any of the benchmarks. [moderate to good] |

### Example 3—Thermogravimetric Analysis (TGA)

Samples were analysed using thermogravimetric analysis with Mettler Toledo TGA\DSC3+ over a temperature ramp from room temperature to 800° C. under air flow.

TABLE 11
Thermogravimetric analysis of benchmark compounds

| | Stability (less than | Mass |
|---|---|---|
| | 2% mass loss) up to | loss at |
| Structure | temp (°C) | 150 °C |
| 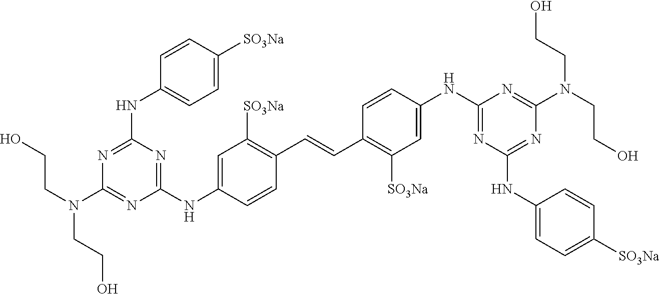 | 79.5 | 7.5% |
| Benchmark 2 (DAS-2) | | |
| CAS 16470-24-9 | | |
| 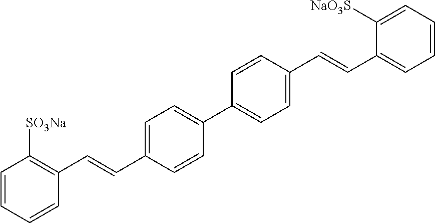 | 78.8 | 7.1% |
| Benchmark 4 (DSBP) | | |
| CAS 27344-41-8 | | |

TABLE 12
Thermogravimetric analysis of example compounds

| | Stability (less than | Mass |
|---|---|---|
| | 2% mass loss) up to | loss at |
| Structure | temp (°C) | 150 ºC |
| 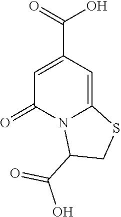 [SMILES: O=C(O)c1cc2n(c(=O)c1)C(C(=O)O)CS2] | 250.0 | 0.1 |
| 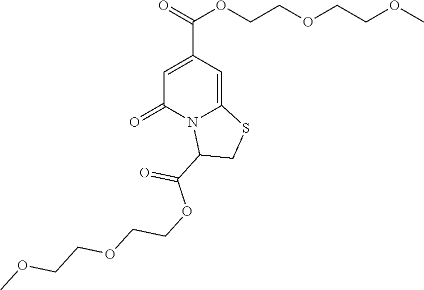 | 129.9 | 3.8% |
| 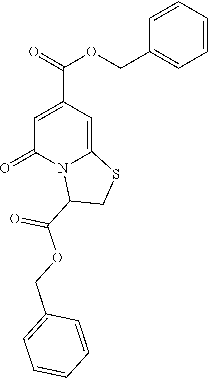 [SMILES: O=C(OCc1ccccc1)c1cc2n(c(=O)c1)C(C(=O)OCc1ccccc1)CS2] | 155.2 | 1.9% |
| 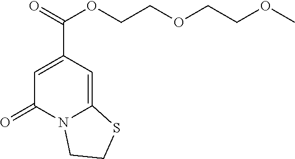 [SMILES: COCCOCCOC(=O)c1cc2n(c(=O)c1)CCS2] | 107.3 | 6.6% |
| 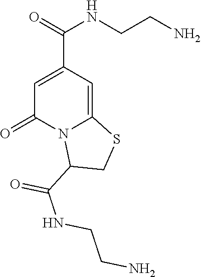 [SMILES: NCCNC(=O)c1cc2n(c(=O)c1)C(C(=O)NCCN)CS2] | 111.6 | 17.2% |
| 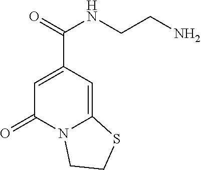 [SMILES: NCCNC(=O)c1cc2n(c(=O)c1)CCS2] | 126.1 | 4.0% |
| 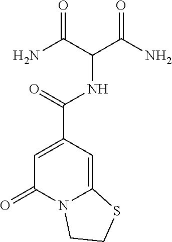 [SMILES: NC(=O)C(NC(=O)c1cc2n(c(=O)c1)CCS2)C(N)=O] | 119.1 | 7.0% |

Some of the low temperature mass loss could be attributed to residual moisture loss. Even so, most compounds of the present invention can be considered stable up to 110° C. They also show comparable mass loss at 150° C. with respect to the benchmarks, so at least a similar degree of thermostability.

Thermal stability of fluorescent whitening agents and optical brighteners of the invention is important as, in the case of laundry powder detergent, the ingredient may be introduced before water removal stage in a fluidised bed dryer where temperatures can reach 50-110° C.

### Example 4—Detergency

Detergent compositions representative of the present invention are described in Tables 13, 14, 15 and 16.

TABLE 13
Representative Laundry Powder Compositions
for Conventional and Compact Compositions
All figures are % by weight.

| Ingredients | Conventional | Compact |
|---|---|---|
| Anionic and non-ionic | 8-15 | 10-30 |
| | | surfactants |
| Builders | 20-50 | 20-40 |
| Cobuilders | 1-5 | 1-7 |
| Bleaching agents | 10-25 | 10-20 |
| Bleach activators | 1-3 | \|2-8 |
| Antiredeposition agents | 1 | 1 |
| Corrosion inhibitors | 2-6 | 2-6 |
| Bleach Stabilizers | 0-1 | 0-1 |
| Foam regulators | 0.1-1 | 0.1-2.0 |
| Enzymes | 0.3-1.0 | 0.5-2.0 |
| FWA or optical brightener | 0.1-0.5 | 0.1-0.5 |

| | |
|---|---|
| Minors, water | balance to 100% |

| | | |
|---|---|---|
| Bulk density, g/L | 500-650 | 600-900 |

TABLE 14
Representative Detergent Tablet Compositions
for Zeolite and Phosphate based Compositions
All figures are % by weight.

| Ingredients | Zeolite-based, % | Phosphate-based, % |
|---|---|---|
| Surfactants | 13-18 | 15-18 |
| Bleaching agents | 13-15 | 12-16 |
| TAED | 3-7 | 4-7 |
| Zeolite 1 | 15-30 | |
| Sodium triphosphate | | 25-45 |
| Layered silicate | 0-9 | 0-9 |
| Sodium polycarboxylate | 2-3 | 2 |
| Disintegrants | 5-17 | 0-12 |
| Enzymes | 2-4 | 2-3 |
| FWA or optical brightener | 0.1-0.5 | 0.1-0.5 |

| | |
|---|---|
| Minors, water | balance to 100% |

TABLE 15
Representative Laundry Powder Compositions
for Homogeneous and Structured Compositions
All figures are % by weight.

| Ingredient | Homogeneous | Structured |
|---|---|---|
| Anionic surfactants | 7-18 | 10-25 |
| Non-ionic surfactants | 15-30 | 6-10 |
| Soaps | 10-25 | 4-8 |
| Builders | 0-5 | 15-30 |
| Solubilizers | 0-12 | 0-5 |
| Alcohols | 5-12 | 0-5 |
| Enzymes | 0-2.5 | 0-1.5 |
| FWA or optical brightener | 0.05-1.0 | 0.05-1.0 |
| Minors, water | balance to 100% | |

TABLE 16
Representative Fabric Bleach Detergent
Compositions for Solid and Liquid Forms
All figures are % by weight.

| Ingredient | Powder | Liquid |
|---|---|---|
| Anionic surfactants | 5-15 | 2-10 |
| Non-ionic surfactants | 0-1 | 3-5 |
| Builders | 60-75 | |
| Bleach | 3-15 | 3-4 |
| Filler | 5-20 | |
| FWA or optical brightener | 0.2-1.0 | 0.1-01.0 |
| Enzymes | 0-1 | |

| | |
|---|---|
| Minors, water | balance to 100% |

1. Use of compounds of formula (I) as fluorescent whitening agents or optical brighteners in a laundry application;
wherein the compounds of formula (I) have the structure: 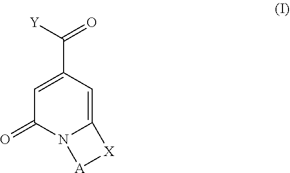
wherein:
A is CHR¹R²(CH₂)ₙ wherein R¹ is H, CH₂OH or COZ, R² is H or CH₂OH and wherein n is 1 or 2, or wherein A is an optionally substituted aromatic or heteroaromatic ring fused with N and X wherein N and X are respectively bound to adjacent carbon atoms on the ring;
X is O, S or N—H;
Y is Z or X′L;
the or each Z is independently OH, OM, OR³, O(CH₂)_{q}SO₃M, NH₂, NHOH, NHR⁴ or NR⁴₂, wherein
M is selected from an alkali metal or an alkaline earth metal;
R³ represents a linear or branched alkyl, an aryl, an alkaryl, an aralkyl, a linear or branched alkyl alcohol, a linear or branched alkyl alcohol polyol, a hydroxyalkylamine, a polyhydric alcohol, a sugar, a linear or branched alkyl ether, a polyester having from 2 to 1000 repeat units, or a polyoxyalkylene chain having from 2 to 1000 repeating units;
q is 1 to 5;
R⁴ is independently selected from methyl, ethyl, propyl, C₄ to C₁₂ alkyl, benzyl, 2-hydroxyethyl, 1-hydroxy-2-propanyl, 1,3-dihydroxy-2-propanyl, 2,3-dihydroxypropyl, cyanomethyl, 2-aminoethyl or dicarbamoylmethyl; alternatively, NR⁴₂ represents a heterocyclic ring; and
wherein when Y is X′L;
X′ is O, S or N—H; and
L represents a linker moiety connecting m repeating units of formula (I) having X′ as defined above in place of Y.

2. Use of the compounds of formula (II) as fluorescent whitening agents or optical brighteners in a laundry application;
wherein the compounds of formula (II) have the structure: 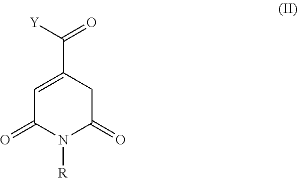
wherein Y is Z or X′L;
Z is OH, OM, OR³, O(CH₂)_{q}SO₃M, NH₂, NHOH, NHR⁴ or NR⁴₂,
M represents an alkali metal or an alkaline earth metal;
R³ represents a linear or branched alkyl, an aryl, an alkaryl, an aralkyl, a linear or branched alkyl alcohol, a linear or branched alcohol polyol, a hydroxyalkylamine, a polyhydric alcohol, a sugar, a linear or branched alkyl ether, a polyester having from 2 to 1000 repeat units, or a polyoxyalkylene chain having from 2 to 1000 repeating units;
q is 1 to 5;
R⁴ is independently selected from methyl, ethyl, propyl, C₄ to C₁₂ alkyl, benzyl, 2-hydroxyethyl, 1-hydroxy-2-propanyl, 1,3-dihydroxy-2-propanyl, 2,3-dihydroxypropyl, cyanomethyl, 2-aminoethyl or dicarbamoylmethyl; alternatively, NR⁴₂ represents a heterocyclic ring; and
R is H, CHR⁵R⁶, R⁵, R⁶, an alkylsulphonate, a polyether, CH₂COZ or CH(COZ)((CH)ₙCOZ) in which each Z may be the same or different,
R⁵ and R⁶ independently represent hydrogen, alkyl (such as methyl, ethyl, propyl, isopropyl), vinyl, phenyl, hydroxymethyl, 1-hydroxyethyl, 2-hydroxyethyl, 1,2-dihydroxyethyl, aminomethyl, (dimethylamino)methyl, 5-aminopentyl, or cyano, optionally wherein R⁵ and R⁶ are the same or different, and
n is 1 or 2;
wherein when Y is X′L;
X′ is O, S or N—H; and
L represents a linker moiety connecting m repeating units of a variant of formula (II) in which X′ as defined above is present in place of Y.

3. The use of compounds of formula (I) according to claim 1, wherein:
a. where R³ represents a linear or branched alkyl, R³ is preferably selected from methyl, ethyl, propyl, butyl, pentyl, hexyl, heptyl, octyl, or 2-ethylhexyl;
b. where R³ represents an alkyl alcohol, R³ is selected from methanol, ethanol, propanol, butanol, pentanol, hexanol, heptanol, octanol, dodecanol, or stearyl or oleyl alcohol;
c. where R³ represents an aryl, R³ is phenyl;
d. where R³ represents an alkaryl, R³ is selected from benzyl, or ethyl phenyl;
e. where R³ represents a linear or branched alkyl alcohol, R³ is selected from 4-hydroxybutyl;
f. where R³ represents a linear or branched alcohol polyol, R³ is a diol or triol, optionally 2,3-dihydroxypropyl, or 2-hydroxy-1-(hydroxymethyl)ethyl;
g. where R³ represents a hydroxyalkylamine, R³ is selected from triethanolamine, N-methyldiethanolamine or triisopropanolamine;
h. where R³ represents a polyhydric alcohol, R³ is glycerol;
i. where R³ represents a sugar, R³ is selected from dextrose, fructose, galactose, glucose, lactose, maltose or sucrose;
j. where R³ represents a linear or branched alkyl ether, R³ is selected from 2-(2-hydroxyethoxy)ethyl, 2-(2-hydroxy-1-methylethoxy)-1-methylethyl, 2-[2-(2-hydroxy-1-methylethoxy)-1-methylethoxy]-1-methylethyl, 3-(3-hydroxypropoxy)propyl, 3-[3-(3-hydroxypropoxy)propoxy]propyl, 4-(4-hydroxybutoxy)butyl, 4-[4-(4-hydroxybutoxy)butoxy]butyl, 2-(2-methoxyethoxy)ethyl, or 2-[2-(2-methoxyethoxy) ethoxy]ethyl; and/or
k. where R³ represents a polyester or a polyoxyalkylene chain having from 2 to 1000 repeat units, the chain is a homopolymer or a copolymer.

4. The use of fluorescent whitening agent or optical brightener compounds according to claim 1 wherein:
a. Linker L is derived from an alcohol, and X′ is the oxygen of the precursor alcohol; or
b. Linker L is derived from an amine, and X′ is the nitrogen of the precursor amine.

5. The use of fluorescent whitening agent or optical brightener compounds according to claim 1, being a dimeric or oligomeric compound wherein m is 2 or more, optionally 3 to 20, optionally 3.

6. The use of fluorescent whitening agent or optical brightener compounds according to claim 1 wherein Linker L is constituted by any of ethylene, propylene or C₄ to C₁₂ alkylene, a polyester chain having from 0 to 1000 repeating units being a homopolymer or copolymer, or a polyoxyalkylene chain having from 0 to about 1000 repeating units being a homopolymer or copolymer, triethanolamine, glycerol or a sugar.

7. The use of fluorescent whitening agent or optical brightener compounds according to claim 1, where the compounds are formulated for use as fluorescent whitening agents, or optical brighteners.

8. The use of fluorescent whitening agent or optical brightener compounds according to claim 7, where the compounds comprise part of a fluorescent whitening or optical brightening formulation, wherein the formulation additionally comprises at least one ancillary compound, optionally selected from one or more of surfactant(s), detergent(s), bleach(es), carrier compound(s), stabilizer(s) and/or dispersant(s).

9. A fluorescent whitening or optical brightening laundry formulation comprising a fluorescent whitening agent or optical brightener compound of formula (I) according to claim 1 and at least one detergent, bleach, carrier compound, stabilizer and/or dispersant;
wherein the compound of formula (I) has the structure: 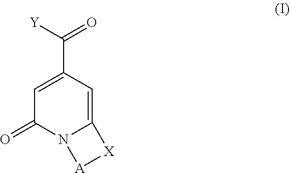
wherein:
A is CHR¹R²(CH₂)ₙ wherein R¹ is H, CH₂OH or COZ, R² is H or CH₂OH and wherein n is 1 or 2, or wherein A is an optionally substituted aromatic or heteroaromatic ring fused with N and X wherein N and X are respectively bound to adjacent carbon atoms on the ring;
X is O, S or N—H;
Y is Z or X′L;
the or each Z is independently OH, OM, OR³, O(CH₂)_{q}SO₃M, NH₂, NHOH, NHR⁴ or NR⁴₂, wherein
M is selected from an alkali metal or an alkaline earth metal;
R³ represents a linear or branched alkyl, an aryl, an alkaryl, an aralkyl, a linear or branched alkyl alcohol, a linear or branched alkyl alcohol polyol, a hydroxyalkylamine, a polyhydric alcohol, a sugar, a linear or branched alkyl ether, a polyester having from 2 to 1000 repeat units, or a polyoxyalkylene chain having from 2 to 1000 repeating units;
q is 1 to 5;
R⁴ is independently selected from methyl, ethyl, propyl, C₄ to C₁₂ alkyl, benzyl, 2-hydroxyethyl, 1-hydroxy-2-propanyl, 1,3-dihydroxy-2-propanyl, 2,3-dihydroxypropyl, cyanomethyl, 2-aminoethyl or dicarbamoylmethyl; alternatively, NR⁴₂ represents a heterocyclic ring; and
wherein when Y is X′L;
X′ is O, S or N—H; and
L represents a linker moiety connecting m repeating units of formula (I) having X′ as defined above in place of Y.

10. A fluorescent whitening or optical brightening laundry formulation comprising a fluorescent whitening agent or optical brightener compound of formula (II) according to claim 2 and at least one detergent, bleach, carrier compound, stabilizer and/or dispersant;
wherein the compound of formula (II) has the structure: 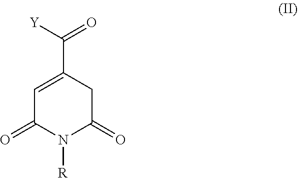
wherein Y is Z or X′L;
Z is OH, OM, OR³, O(CH₂)_{q}SO₃M, NH₂, NHOH, NHR⁴ or NR⁴₂,
M represents an alkali metal or an alkaline earth metal;
R³ represents a linear or branched alkyl, an aryl, an alkaryl, an aralkyl, a linear or branched alkyl alcohol, a linear or branched alcohol polyol, a hydroxyalkylamine, a polyhydric alcohol, a sugar, a linear or branched alkyl ether, a polyester having from 2 to 1000 repeat units, or a polyoxyalkylene chain having from 2 to 1000 repeating units;
q is 1 to 5;
R⁴ is independently selected from methyl, ethyl, propyl, C₄ to C₁₂ alkyl, benzyl, 2-hydroxyethyl, 1-hydroxy-2-propanyl, 1,3-dihydroxy-2-propanyl, 2,3-dihydroxypropyl, cyanomethyl, 2-aminoethyl or dicarbamoylmethyl; alternatively, NR⁴₂ represents a heterocyclic ring; and
R is H, CHR⁵R⁶, R⁵, R⁶, an alkylsulphonate, a polyether, CH₂COZ or CH(COZ)((CH)ₙCOZ) in which each Z may be the same or different,
R⁵ and R⁶ independently represent hydrogen, alkyl (such as methyl, ethyl, propyl, isopropyl), vinyl, phenyl, hydroxymethyl, 1-hydroxyethyl, 2-hydroxyethyl, 1,2-dihydroxyethyl, aminomethyl, (dimethylamino)methyl, 5-aminopentyl, or cyano, optionally wherein R⁵ and R⁶ are the same or different, and
n is 1 or 2;
wherein when Y is X′L;
X′ is O, S or N—H; and
L represents a linker moiety connecting m repeating units of a variant of formula (II) in which X′ as defined above is present in place of Y.

11. A method for providing fluorescent whitening or optical brightening to a laundry substrate comprising contacting the laundry substrate with a fluorescent whitening agent or optical brightener compound of formula (I) or a fluorescent whitening or optical brightening formulation according to claim 9 under conditions effective to allow chemical and/or physical bonding of the fluorescent whitening agent or optical brightener onto or into the substrate;
wherein the compound of formula (I) has the structure: 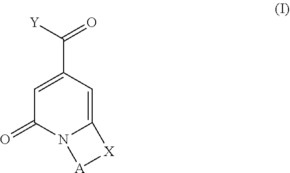
wherein:
A is CHR¹R²(CH₂)ₙ wherein R¹ is H, CH₂OH or COZ, R² is H or CH₂OH and wherein n is 1 or 2, or wherein A is an optionally substituted aromatic or heteroaromatic ring fused with N and X wherein N and X are respectively bound to adjacent carbon atoms on the ring;
X is O, S or N—H;
Y is Z or X′L;
the or each Z is independently OH, OM, OR³, O(CH₂)_{q}SO₃M, NH₂, NHOH, NHR⁴ or NR⁴₂, wherein
M is selected from an alkali metal or an alkaline earth metal;
R³ represents a linear or branched alkyl, an aryl, an alkaryl, an aralkyl, a linear or branched alkyl alcohol, a linear or branched alkyl alcohol polyol, a hydroxyalkylamine, a polyhydric alcohol, a sugar, a linear or branched alkyl ether, a polyester having from 2 to 1000 repeat units, or a polyoxyalkylene chain having from 2 to 1000 repeating units;
q is 1 to 5;
R⁴ is independently selected from methyl, ethyl, propyl, C₄ to C₁₂ alkyl, benzyl, 2-hydroxyethyl, 1-hydroxy-2-propanyl, 1,3-dihydroxy-2-propanyl, 2,3-dihydroxypropyl, cyanomethyl, 2-aminoethyl or dicarbamoylmethyl; alternatively, NR⁴₂ represents a heterocyclic ring; and
wherein when Y is X′L;
X′ is O, S or N—H; and
L represents a linker moiety connecting m repeating units of formula (I) having X′ as defined above in place of Y.

12. A method for providing fluorescent whitening or optical brightening to a laundry substrate comprising contacting the laundry substrate with a fluorescent whitening agent or optical brightener compound of formula (II) or a fluorescent whitening or optical brightening formulation according to claim 10 under conditions effective to allow chemical and/or physical bonding of the fluorescent whitening agent or optical brightener onto or into the substrate;
wherein the compound of formula (II) has the structure: 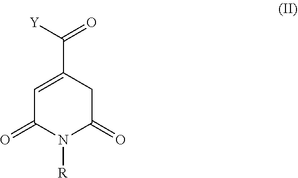
wherein Y is Z or X′L;
Z is OH, OM, OR³, O(CH₂)_{q}SO₃M, NH₂, NHOH, NHR⁴ or NR⁴₂,
M represents an alkali metal or an alkaline earth metal;
R³ represents a linear or branched alkyl, an aryl, an alkaryl, an aralkyl, a linear or branched alkyl alcohol, a linear or branched alcohol polyol, a hydroxyalkylamine, a polyhydric alcohol, a sugar, a linear or branched alkyl ether, a polyester having from 2 to 1000 repeat units, or a polyoxyalkylene chain having from 2 to 1000 repeating units;
q is 1 to 5;
R⁴ is independently selected from methyl, ethyl, propyl, C₄ to C₁₂ alkyl, benzyl, 2-hydroxyethyl, 1-hydroxy-2-propanyl, 1,3-dihydroxy-2-propanyl, 2,3-dihydroxypropyl, cyanomethyl, 2-aminoethyl or dicarbamoylmethyl; alternatively, NR⁴₂ represents a heterocyclic ring; and
R is H, CHR⁵R⁶, R⁵, R⁶, an alkylsulphonate, a polyether, CH₂COZ or CH(COZ)((CH)ₙCOZ) in which each Z may be the same or different,
R⁵ and R⁶ independently represent hydrogen, alkyl (such as methyl, ethyl, propyl, isopropyl), vinyl, phenyl, hydroxymethyl, 1-hydroxyethyl, 2-hydroxyethyl, 1,2-dihydroxyethyl, aminomethyl, (dimethylamino)methyl, 5-aminopentyl, or cyano, optionally wherein R⁵ and R⁶ are the same or different, and
n is 1 or 2;
wherein when Y is X′L;
X′ is O, S or N—H; and
L represents a linker moiety connecting m repeating units of a variant of formula (II) in which X′ as defined above is present in place of Y.
